(19) United States
(12) Patent Application Publication
(10) Pub. No.: US 2008/0138404 A1
(43) Pub. Date: Jun. 12, 2008

(54) EXTENDED RELEASE FORMULATIONS OF CARVEDILOL

(75) Inventors: Edwin WALSH, Dublin 3 (IE); Ann Marie YOUNG, Co. Kildare (IE); Graham JACKSON, Co. Kildare (IE)

Correspondence Address: OBLON, SPIVAK, MCCLELLAND MAIER & NEUSTADT, P.C., 1940 DUKE STREET, ALEXANDRIA, VA, 22314, US

(73) Assignee: BIOVAIL LABORATORIES INTERNATIONAL S.R.L., St. Michael (BB)

(21) Appl. No.: 11/945,422

(22) Filed: Nov. 27, 2007

Related U.S. Application Data
(60) Provisional application No. 60/868,880, filed on Dec. 6, 2006.

(51) Int. Cl. A61K 9/22; A61K 31/403; A61K 9/52

(52) U.S. Cl. 424/452; 514/411; 424/465

(57) ABSTRACT

An improved controlled release dosage form for once-daily administration of carvedilol is described. The controlled release dosage form comprises a therapeutically effective amount of carvedilol and/or a pharmaceutically acceptable salt thereof; one or more hydrophilic polymers; one or more pharmaceutically acceptable excipients; and a polyoxyalkylene block copolymer, a solid dispersion of carvedilol and an extrusion material or a combination of a polyoxyalkylene block copolymer, a solid dispersion of carvedilol and an extrusion material.

### FIELD OF THE INVENTION

The present invention relates to improved dosage forms for once-daily administration of carvedilol. The controlled release dosage forms of carvedilol according to the invention can be suitable for gastric retention. The present invention also relates to the use of such dosage forms for the treatment of one or more conditions such as cardiovascular disorders, for example, in a subject suitable for treatment by carvedilol or pharmaceutically acceptable salts thereof.

### BACKGROUND TO THE INVENTION

Carvedilol is a beta-adrenergic receptor blocking drug with ancillary vasodilatory properties. The current commercial formulation for carvedilol is immediate release, and is administered twice daily. The immediate release formulation of carvedilol is rapidly and extensively absorbed following oral administration, with a terminal half-life ranging from 7-10 hours. A once-daily dosing formulation for carvedilol is commercially desirable, would reduce a patient's dosing regimen and can improve patient compliance.

Carvedilol (1-(carbazol-4-yloxy-3-[[2-(o-methoxyphenoxy)ethyl]-amino]-2-propanol) is disclosed in U.S. Pat. No. 4,503,067 to Wiedermann et al, issued Mar. 5, 1985. Carvedilol is currently synthesized as a free base with a molecular weight of 406.5 and a molecular formula of C₂₄H₂₆N₂O₄. The original commercially available carvedilol containing drug product, Coreg®, is a conventional white, oval, film coated release tablet containing 3.125 mg, 6.25 mg, 12.5 mg or 25 mg of carvedilol, prescribed as a twice-a-day medication. Coreg® is an immediate release or rapidly releasing formulation, where the chemical and physical formulation properties are such that by the time the carvedilol leaves the stomach, it is either in solution or it is in the form of a suspension of fine particles such that it can be readily absorbed (Choon et al, 2004)).

Carvedilol contains an α-hydroxyl secondary amine, with a pKa of 7.8. It exhibits predictable solubility behaviour in neutral or alkaline media, i.e. above a pH of 9.0 the solubility of carvedilol is relatively low (<1 μg/ml). The solubility of carvedilol increases with decreasing pH and reaches a plateau near pH 5 where saturation solubility is about 23 μg/ml at pH 7 and about 100 μg/ml at pH 5 at room temperature. At lower pH values the solubility of carvedilol is limited by the solubility of its protonated form or its corresponding salt formed in-situ.

Low-solubility drugs, such as carvedilol often show poor bioavailability or irregular absorption. The degree of irregularity of absorption is affected by factors such as dose level, the fed state of the patient, and form of the drug. A large amount of research has been carried out in relation to methods of increasing the bioavailability of low-solubility drugs. In order to increase the bioavailability of a drug it is necessary to improve the concentration of the drug in solution in order to improve absorption.

Carvedilol is an arlethanolamine synthesized as a free base racemic mixture of 2 enantiomers. The S(−) enantiomer possesses β-adrenoceptor blocking activity, while the racemate also has α₁-receptor blocking activity due to the activity of the R(+) enantiomer. As a result carvedilol possesses two complementary pharmacologic actions—mixed venous and arterial vasodilation and non-cardioselective β-adrenergic blockade. Reviews on the detailed pharmacodynamic and therapeutic properties of carvedilol are available in the literature (Morgan (1994), McTavish et al (1993)).

Carvedilol is rapidly absorbed when administered orally with maximum plasma concentrations (Cmax) reached within 1 to 2 hours (Tmax) in both healthy volunteers and hypertensive patients. Studies have demonstrated that peak plasma concentrations of carvedilol increase linearly with dose (between 12.5 mg and 50 mg) and that absorption is not altered following repeated immediate release doses of carvedilol. Further, little accumulation of carvedilol has been observed following multiple immediate release doses of carvedilol, as indicated by similar mean AUC in comparison with a single dose administration (McPhillips et al (1988), Morgan et al (1990)). The rate of absorption of carvedilol has been shown to decrease marginally when taken as an immediate release dose with food, but the extent of absorption is unaffected (Louis et al, 1987). Carvedilol reportedly possesses an absolute bioavailability of approximately 25% to 35% due to a significant degree of stereo-selective first pass metabolism (Coreg® Prescribing Information, von Mollendorff et al (1987)) with plasma levels of the R(+) Carvedilol approximately 2 to 3 times higher than S(−) and less than 2% of an immediate release dose recovered as unchanged drug in urine. The primary P450 enzymes responsible for metabolism of both enantiomers are CYP2D6 and CYP2C9 and to a much lesser extent CYP3A4. Carvedilol is subject to significant genetic polymorphism with poor metabolisers showing 2 to 3-fold higher plasma concentration of R(+)Carvedilol compared to the extensive metabolisers. Plasma levels of S(−) Carvedilol are only increased by about 20% to 25% in poor metabolisers. Following oral administration in healthy subjects Carvedilol is more than 98% bound to plasma protein, primarily with albumin. The extent of plasma-protein binding is independent of concentration over the therapeutic range. Following oral administration, the apparent mean terminal elimination half-life of carvedilol generally ranges from 7 to 10 hours.

When developing an extended release carvedilol formulation, it is important to consider the gastrointestinal (GI) regional absorption of the drug substance. It is reported (WO 2003/028718), based upon studies performed by Boehringer Mannheim in man with a 25 mg carvedilol containing gelatin suspension, that the colonic absorption of carvedilol is about 7% of that following oral administration. The relative absorption of carvedilol in the jejunum and ileum is reported as 56% and 28% respectively in comparison with oral administration. Further, data presented by Nolte et al (1999) using the in-vitro porcine intestine based Boehringer-Mannheim ring model (BM-RIMO) suggests that carvedilol absorption is mediated through the transcellular route and decreases within the GI tract in the order jejunum, ileum, colon.

The practically insoluble nature of carvedilol and potentially narrow absorption window reported for carvedilol presents a significant challenge when selecting an appropriate drug delivery platform to facilitate the development of a once-daily extended release formulation. Conventional diffusion systems based upon drug release through a water permeable film coated tablet or multiparticulate are likely to demonstrate incomplete drug release over time due to unfavourable drug solubility.

Formulations based on gastric retention drug delivery mechanisms have been described that are suitable for delivery of drugs which are poorly soluble and possess poor relative bioavailability in the distal small intestine and large intestine.

WO 2005/079752 describes a controlled release oral pharmaceutical composition having a therapeutically effective amount of one or more pharmacologically active agents having low bioavailability, one or more solubilizers; one or more biocompatible swelling agents; and a swelling enhancer. The swelling agent, in combination with swelling enhancer, swells in the presence of water in gastric fluid such that the size of the dosage form is sufficiently increased to provide retention of the dosage form in the stomach of a patient, which gradually erodes within the gastrointestinal tract over a prolonged time period. However WO 2005/079752 does not disclose any in-vivo data that demonstrates that the invention is suitable for controlled delivery of once-a-day administration of a drug substance.

U.S. Pat. No. 6,723,340 describes unit dosage forms for drugs that benefit from a prolonged time of controlled release in the stomach and upper gastrointestinal (GI) tract, and from an enhanced opportunity for absorption in the stomach and upper GI tract rather than the lower portions of the GI tract. The dosage forms described are suitable for gastric retention and comprise the active ingredient dispersed in a solid unitary matrix that is formed of a combination of poly(ethylene oxide) and hydroxypropyl methylcellulose. By selecting an appropriate combination of polymers and appropriate grade of each polymer, an appropriate balance between tablet swelling and tablet erosion is achieved, whereby the release of a drug with solubility less than 1 part drug in ten parts water can be achieved from a dosage form maintained in the stomach lumen as a result of tablet swelling. However, U.S. Pat. No. 6,723,340 does not demonstrate the ability of the technology described to enable controlled release of a drug with solubility below approximately 0.5 parts drug in 10 parts water, in particular below approximately 0.3 parts drug to 10 parts water.

U.S. Pat. No. 6,391,338 discloses a method for enhancing the solubility of a substantially water-insoluble bio-affecting agent in an aqueous environment of a bio-system. The method described involves transforming said agent into a solid substantially uniform dispersion with a water-soluble polymer. However, it does not disclose the use of solid dispersions in gastric retention delivery systems.

U.S. Pat. No. 6,117,452 discloses a method of preparing thermoformed particulates of active agents via processes, which employ certain combinations of fatty esters and optional surfactants or emulsifiers as processing aids. The compositions resulting from the disclosed process contain one or more active agents, a combination of processing aids consisting essentially of glyceryl monostearate, and polyethylene glycol (32) glyceryl palmitylstearate, and one or more optional emulsifiers and/or surfactants. However, it does not disclose compositions for use in gastric retention delivery systems.

US 2005/0019339 relates to pharmaceutical compositions in which carvedilol is present at least partially in its amorphous form in a solid dispersion together with a plasticizing polymer such as polyethylene oxide in combination with a stabilizer. The solid dispersion is further formulated into a cylindrical ‘Egalet®’ dosage form in which only the open ends are exposed to the external environment, thus creating a dosage form capable of near zero order release kinetics. Based upon the pharmacokinetic data presented a Tmax of only 4 hours is achievable using this formulation approach.

US 2004/0185105 A1 discloses gastric retention formulations in which the dosage form is retained in the upper GI tract of a patient when in the fed state. The invention relies upon matrix tablets comprising hydrophilic, swellable and erodible polymers in combination with the active drug. The polymers hydrate and swell to a size sufficient to withstand expulsion through the pyloric sphincter of the stomach. As a result, drug released from the dosage form as a result of diffusion and erosion will be absorbed as preferred in the proximal intestine, and conversely, little drug will be exposed to the colon. Such a formulation approach will ideally employ high molecular weight or high viscosity hydrophilic polymers to ensure rapid hydration and therefore rapid swelling to reduce the likelihood of tablet expulsion from the stomach. However, for poorly soluble drugs such as carvedilol, the use of such polymers is likely to present such a significant diffusion barrier that adequate drug release can be prevented. The use of lower molecular weight or viscosity polymers can enable better drug release by erosion, but can not provide the properties, both swelling potential and also mechanical resistance to hydrodynamic shear in the lumen of the stomach in the fed state, to withstand tablet disintegration and unwanted premature drug release.

There is therefore a need for an improved extended release dosage form of carvedilol which addresses the potential limitation of swellable, gastrically retained dosage forms for delivery of poorly water soluble drugs to the proximal small intestine.

It is therefore an object of the present invention to provide an improved extended release formulation of carvedilol that allows adequate rate and release of drug both in the stomach and small intestine.

### SUMMARY OF THE INVENTION

Accordingly, the invention provides a controlled release dosage form, said dosage form comprising a therapeutically effective amount of carvedilol and/or a pharmaceutically acceptable salt thereof; one or more hydrophilic polymers; one or more pharmaceutical excipients and a polyoxyalkylene block copolymer.

In an alternative embodiment the controlled release dosage form comprises a therapeutically effective amount of carvedilol and/or a pharmaceutically acceptable salt thereof; one or more hydrophilic polymers; one or more pharmaceutically acceptable excipients, and a solid dispersion of carvedilol and an extrusion material.

In yet another embodiment the controlled release dosage form comprises a therapeutically effective amount of carvedilol and/or a pharmaceutically acceptable salt thereof; one or more hydrophilic polymers; one or more pharmaceutically acceptable excipients, a polyoxyalkylene block copolymer and a solid dispersion of carvedilol and an extrusion material.

In still another embodiment the invention provides a controlled release dosage form, said dosage form comprising a therapeutically effective amount of carvedilol and/or a pharmaceutically acceptable salt thereof; one or more hydrophilic polymers; one or more pharmaceutically acceptable excipients; and means for enhancing the rate and/or extent of release of carvedilol from said dosage form.

In one embodiment the dosage form according to the invention comprises a gastrically retainable dosage form.

In one embodiment, the dosage form according to the invention provides a gastrically retainable formulation for delivery of carvedilol to the upper GI tract, which demonstrates an improved extent of release of carvedilol compared to known dosage forms. The dosage form according to the invention also demonstrates an improved rate of release of carvedilol from the dosage form.

In one aspect of the invention, the means for enhancing the rate and/or extent of release of carvedilol comprises a polyoxyalkylene block copolymer.

In another aspect of the invention, the means for enhancing the rate and/or extent of release of carvedilol comprises a solid dispersion of carvedilol and an extrusion material.

In another aspect of the invention, the carvedilol comprises a solid dispersion of carvedilol.

In one aspect of the invention said extrusion material comprises an extrusion polymer.

The extrusion polymer can be selected from the group consisting of a methacrylic acid ester terpolymeric product of butyl methacrylate, (2-dimethyl aminoethyl)methacrylate, and methyl methacrylate 1:2:1 (EUDRAGIT® E), polyethylene glycols, polyoxyethylene glycols, polyethylene-propylene glycol copolymers, polyethylene oxides, polyvinyl pyrrolidinone (also referred to as polyvinyl pyrrolidone or povidone or PVP), polyvinyl alcohol, polyethylenevinyl alcohol copolymers, polyvinyl alcohol polyvinyl acetate copolymers, xanthan gum, carrageenan, hydroxypropyl cellulose, hydroxypropyl methylcellulose, carboxymethyl cellulose, carboxymethyl ethyl cellulose, carboxylic acid-functionalized polymethacrylates, amine-functionalized polymethacrylates, chitosan, chitin, polydextrose, dextrin and starch, and any combination thereof.

The skilled person will appreciate that polymeric materials such as high molecular weight proteins such as gelatin and albumin could also be used in accordance with the present invention. Examples of polyethylene glycols and polyoxyethylene glycols include the CARBOWAX®7 polymers supplied by Union Carbide (Danbury, Conn.) and the LUTROL® E polymers supplied by BASF (Mount Olive, N.J.). Examples of polyethylene oxide include POLOX® supplied by Union Carbide. Examples of polyvinyl pyrrolidinones include the KOLLIDON® polymers supplied by BASF. Examples of polyvinyl alcohols and polyvinyl alcohol polyvinyl acetate copolymers include the ELVANOL® polymers supplied by DuPont Industrial Polymers (Wilmington Del.). Examples of polyethylenevinyl alcohol copolymers include the EV AL® polymers supplied by EV ALCA (Lisle, Ill.). Examples of xanthan gums include the KETROL® polymers supplied by Monsanto Pharmaceutical Ingredients (St. Louis, Mo.).

Examples of carrageenans include the GELCAREN® polymers supplied by FMC (Philadelphia, Pa.). Examples of hydroxypropyl cellulose include the KLUCEL® polymers supplied by Aqualon Division of Hercules (Wilmington, Del.). Examples of hydroxypropyl methyl cellulose include the Methocel J polymers manufactured by Dow Chemical (Midland, Mich.). Examples of carboxymethyl cellulose include the AKUCEL® polymers supplied by Robeco Inc. (New York. N.Y.). Examples of polydextrose include the LITESSE® polymers supplied by Cultor Food Science (Ardsley, N.Y.).

In one aspect of the invention the extrusion polymer comprises a methacrylic acid ester terpolymeric product of butyl methacrylate, (2-dimethyl aminoethyl)methacrylate, and methyl methacrylate 1:2:1 (EUDRAGIT® E).

Eudragit® E is a copolymer based on dimethylaminoethyl methacrylate and other neutral methacrylic acid esters and marketed by Rohm GmbH. This polymer is available in solvent free granules (EUDRAGIT® E 100) and in a 12.5% solution in propan-2-ol/acetone (60:40) (EUDRAGIT® E 12.5). EUDRAGIT® E has high aqueous solubility especially under acidic conditions (below pH 5) and provides for rapid release of the drug in the gastric region of the gastrointestinal tract.

In one embodiment of the invention the ratio of carvedilol:methyl methacrylate 1:2:1 (Eudragit® E) is 40:60.

In another embodiment of the invention the ratio of carvedilol:methyl methacrylate 1:2:1 (Eudragit® E) is 30:70.

In further embodiment of the invention the ratio of carvedilol: methyl methacrylate 1:2:1 (Eudragit® E) is 20:80.

In an alternative embodiment said extrusion material comprises a non-polymeric material. The solid dispersion of carvedilol can also be formed through the use of a non-polymeric material. By “non-polymeric” is meant that the component is not polymeric. Exemplary non-polymeric materials for use in the formation of a solid dispersion include: alcohols, such as stearyl alcohol and cetyl alcohol, organic acids. such as stearic acid. citric acid, fumaric acid, tartaric acid. and malic acid; organic bases such as glucosamine, N-methylglucamine. tris (hydroxymethyl)amino methane, and dodecylamine; salts such as sodium chloride, potassium chloride, lithium chloride, calcium chloride, magnesium chloride, sodium sulfate, potassium sulfate, sodium carbonate, and magnesium sulfate; amino acids such as alanine and glycine; sugars such as glucose, sucrose, xylitol, fructose, lactose, mannitol, sorbitol, and maltitol; fatty acid esters such as glyceryl (mono- and di-) stearates, glyceryl (mono- and di-) behenates. triglycerdes, sorbitan monostearate, saccharose monostearate, glyceryl (palmitic stearic) ester, polyoxyethylene sorbitan fatty-acid esters; waxes, such as microcrystalline wax, paraffin wax, beeswax, synthetic wax, castor wax, and carnauba wax; alkylsulfates such as sodium lauryl sulfate and magnesium lauryl sulfate; and phospholipids, such as lecithin.

In a further aspect of the invention the means for enhancing the rate and/or extent of release of carvedilol comprises a combination of polyoxyalkylene block copolymer and a solid dispersion of carvedilol and an extrusion polymer.

The one or more hydrophilic polymers used in accordance with the invention can be selected from the group consisting of polyethylene oxide, hydroxypropyl methylcellulose, hydroxyethyl cellulose, hydroxypropyl methylcellulose, hydroxypropyl methylcellulose phthalate and methylcellulose.

In one aspect of the invention the dosage form comprises at least two hydrophilic polymers.

The dosage form according to the invention can comprise hydrophilic polymers having a high molecular weight and/or high viscosity. The use of high molecular weight and/or high viscosity hydrophilic polymers ensures rapid hydration and therefore rapid swelling of the dosage form to reduce the likelihood of expulsion of the dosage form from the stomach.

In one aspect of the invention said hydrophilic polymers comprise polyethylene oxide and hydroxypropyl methylcellulose.

In one aspect of the invention the polyethylene oxide (PEO) has a molecular weight in the range 900,000 to 10,000,000. The skilled person will appreciate that different grades of PEO can be used in the dosage form according to the invention. For example, Polyox™ PEO WSR N-60K having a molecular weight of 2,000,000 or Polyox™ PEO WSR Coagulant (supplied by Dow Chemical Company) having a molecular weight of 5,000,000 can be used.

In one aspect of the invention, the hydroxypropyl methylcellulose (HPMC) has a viscosity in the range from about 4,000 centipoise to about 1,000,000 centipoise when measured as a 2% solution in water at 20° C. The skilled person will appreciate that different grades of HPMC having high viscosity such as Methocel K100M, Methocel K15M and Methocel K4M (supplied by Colorcon).

In one embodiment of the invention said polyoxyalkylene block copolymer comprises a block copolymer of ethylene oxide:propylene oxide:ethylene oxide. This family of copolymers comprises a:b:a block co-polymers of ethylene oxide:propylene oxide:ethylene oxide. The “a” and “b” represent the average number of monomer units for each block of the polymer chain.

In one aspect of the invention said polyoxyalkylene block copolymer comprises an α-hydro-ω-hydroxypoly(oxyethylene)poly(oxypropylene) poly(oxyethylene) block copolymer.

These surfactants are commercially available from BASF Corporation of Mount Olive, N.J., in a variety of different molecular weights and with different values of “a” and “b” blocks. For example, Pluronic® F127 (Poloxamer 407) has a molecular weight range of 9,840 to 14,600 and where “a” is approximately 101 and “b” is approximately 56, Pluronic® F68 (Poloxamer 188) represents an average molecular weight of 7,680 to 9,510 where “a” has a value of about 80 and “b” has a value of about 27, Pluronic® F87 (Poloxamer 237) represents an average molecular weight of 6,840 to 8,830 where “a” has a value of about 64 and “b” has a value of about 37, and Pluronic® F108 (Poloxamer 338) represents an average molecular weight of 12,700 to 17,400 where “a” has a value of about 141 and “b” has a value of about 44. Accordingly, the polyoxyalkylene block copolymer can be selected from the group consisting of HO(C₂H₄O)₈₀(C₃H₆O)₂₇(C₂H₄O)₈₀H [Pluronic® F68 (Poloxamer 188)], HO(C₂H₄O)₁₀₁(C₃H₆O)₅₆(C₂H₄O)₁₀₁H [Pluronic® F127 (Poloxamer 407)], HO(C₂H₄O)₆₄(C₃H₆O)₃₇(C₂H₄O)₆₄H [Pluronic® F87 (Poloxamer 237)], HO(C₂H₄O)₁₄₁(C₃H₆O)₄₄(C₂H₄O)₁₄₁H [Pluronic®F108 (Poloxamer 338)].

The skilled person will appreciate that other copolymers could be used in the dosage form according to the present invention. For example, other acceptable copolymers include, for example, a surfactant of polyoxyl 40 stearate and polyoxyl 50 stearate. Other copolymers that could be used include those that are solids at room temperature and include members selected from the group essentially consisting of sorbitan monopalmitate, sorbitan monostearate, glycerol monostearate and polyoxyethlene stearate (self emulsifying), polyoxyethylene 40 sorbitol lanolin derivative, polyoxyethylene 75 sorbitol lanolin derivative, polyoxyethylene 6 sorbitol beeswax derivative, polyoxyethylene 20 sorbitol beeswax derivative, polyoxyethylene 20 sorbitol lanolin derivative, polyoxyethylene 50 sorbitol lanolin derivative, polyoxyethylene 23 lauryl ether, polyoxyethylene 23 lauryl ether with butylated hydroxyanisole and citric acid added as preservatives, polyoxyethylene 2 cetyl ether with butylated hydroxyanisole and citric acid added as preservatives, polyoxyethylene 10 cetyl ether with butylated hydroxyanisole and citric acid added as preservatives, polyoxyethylene 20 cetyl ether with butylated hydroxyanisole and citric acid added as preservatives, polyoxyethylene 2 stearyl ether with butylated hydroxyanisole and citric acid added as preservatives, polyoxyethylene 10 stearyl ether with butylated hydroxyanisole and citric acid added as preservatives, polyoxyethylene 20 stearyl ether with butylated hydroxyanisole and citric acid added as preservatives, polyoxyethylene 21 stearyl ether with butylated hydroxyanisole and citric acid added as preservatives, polyoxyethylene 20 oleyl ether with butylated hydroxyanisole and citric acid added as preservatives, polyoxyethylene 40 stearate, polyoxyethylene 50 stearate, polyoxyethylene 100 stearate, sorbitan monopalmitate, sorbitan monostearate, sorbitan tristearate, polyoxyethylene 4 sorbitan monostearate, polyoxyethylene 20 sorbitan tristearate, and the like. A resource of surfactants including solid surfactants and their properties is available in McCutcheon's Detergents and Emulsifiers, International Edition 1979 and McCutcheon's Detergents and Emulsifiers, North American Edition 1979. Other sources of information on properties of solid surfactants include BASF Technical Bulletin Pluronic® & Tetronic® Surfactants 1999 and General Characteristics of Surfactants from ICI Americas Bulletin 0-1 10/80 5M. One of the characteristics of surfactants tabulated in these references is the HLB value, or hydrophilic lipophilic balance value. This value represents the relative hydrophilicity and relative hydrophobicity of a surfactant molecule. Generally, the higher the HLB value, the greater the hydrophilicity of the surfactant while the lower the HLB value, the greater the hydrophobicity. For the Pluronic® molecules, for example, the ethylene oxide fraction represents the hydrophilic moiety and the propylene oxide fraction represents the hydrophobic fraction. The HLB values of Pluronic® F127, F87, F108, and F68 are respectively 22.0, 24.0, 27.0, and 29.0.

The combination of polyoxyalkylene block copolymer with both high viscosity HPMC and high molecular weight PEO grades enables release of drug from a dosage form designed for gastric retention. The formulation according to one aspect of the invention is designed such that the inclusion of an agent to improve the hydration rate of the dosage form, such as a tablet, for example, does not compromise the integrity of the dosage form, which is critical to ensure gastric retention and controlled drug delivery.

In one embodiment of the invention the controlled release dosage form comprises a crystalline form of carvedilol.

In one embodiment of the invention the controlled release dosage form comprises an amorphous form of carvedilol.

In one embodiment of the invention the controlled release dosage form comprises a combination of an amorphous and crystalline form of carvedilol.

In an alternative aspect of the invention the carvedilol is at least partially in amorphous form. In the aspect of the invention wherein the means for enhancing the rate and/or extent of release of carvedilol comprises a solid dispersion of carvedilol and extrusion polymer, the carvedilol is present in at least partially amorphous form.

The skilled person will appreciate that the amorphous form of a low-solubility drug, such as carvedilol, that is capable of existing in either the crystalline or amorphous form can temporarily provide a greater aqueous concentration of drug relative to the equilibrium concentration obtained by dissolution of drug in the environment of use. In accordance with one aspect of the invention, such amorphous form comprises a dispersion of the drug in a matrix material. It is believed that such amorphous forms of the drug can dissolve more rapidly than the crystalline form, often dissolving faster than the drug can precipitate from solution. As a result, the amorphous form can temporarily provide a greater than equilibrium concentration of drug.

The dosage form according to the invention can comprise a diluent selected from the group consisting of lactose, microcrystalline cellulose (Avicel® pH101), powdered cellulose, cellulose acetate, silicified microcrystalline cellulose, carboxymethylcellulose calcium, calcium phosphate, calcium sulfate, hydrogenated vegetable oils, sugars such as sucrose, dextrose, maltose and lactose; glyceryl palmitostearate, pregelatinised starch, sorbitol and maltitol.

The dosage form according to the invention can comprise a lubricant selected from the group consisting of magnesium stearate, sodium stearyl fumarate, colloidal silicon dioxide, glyceryl monostearate, glyceryl dibehanate and talc.

The skilled person will appreciate that the dosage form can be in any form suitable for oral administration such as tablets, capsules and coated tablets, for example.

In one aspect of the invention the controlled release dosage form can comprise a tablet.

In another aspect of the invention the dosage form can comprise a capsule.

In one aspect the invention provides a controlled release dosage form comprising

carvedilol in an amount from about 0.1% to about 50% by weight;
PEO in an amount from about 1% to about 60% by weight;
HPMC in an amount from about 1% to about 60% by weight;
Lactose in an amount from about 0% to about 80% by weight;
Microcrystalline cellulose in an amount from about 1% to about 99% by weight;
Magnesium stearate in an amount from about 0.05% to about 10% by weight;
and polyalkylene block copolymer in an amount from about 5% to about 40% by weight.

The carvedilol can be present in an amount selected from about 0.1%, about 0.5%, about 1%, about 5%, about 10%, about 15%, about 20%, about 25%, about 30%, about 35%, about 40%, about 45%, or about 50% by weight.

The PEO can be present in an amount selected from about 1%, about 5%, about 10%, about 15%, about 20%, about 25%, about 30%, about 35%, about 40%, about 45%, about 50%, about 55% or about 60% by weight.

The HPMC can be present in an amount selected from about 1%, about 5%, about 10%, about 15%, about 20%, about 25%, about 30%, about 35%, about 40%, about 45%, about 50%, about 55% or about 60% by weight.

The lactose can be present in an amount selected from about 0%, about 5%, about 10%, about 15%, about 20%, about 25%, about 30%, about 35%, about 40%, about 45%, about 50%, about 55%, about 60%, about 65%, about 70%, about 75% or about 80% by weight.

The microcrystalline cellulose can be present in an amount selected from about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99% by weight.

The magnesium stearate can be present in an amount selected from about 0.05%, about 0.1%, about 1%, about 5% or about 10% by weight.

The polyalkylene block copolymer can be present in an amount selected from about 5%, about 10%, about 15%, about 20%, about 25%, about 30%, about 35% or about 40% by weight.

In the embodiment of the invention where the dosage form is uncoated, the amounts stated herein represent the amount by weight of the overall dosage form for each component. For those aspects of the invention where the dosage form is coated, the amounts of each component stated herein represent the amounts present in the core.

In another aspect the invention provides a controlled release dosage form comprising carvedilol in an amount of about 7.7% by weight;

PEO in an amount of about 10% by weight;
HPMC in an amount of about 10% by weight;
Lactose in an amount of about 25.65% by weight;
Microcrystalline cellulose in an amount of about 25.65% by weight;
Magnesium stearate in an amount of about 1% by weight;
and polyalkylene block copolymer in an amount of about 20% by weight.

In an alternative embodiment the invention provides a controlled release dosage form comprising carvedilol in an amount from about 0.1% to about 50% by weight; Eudragit® E in an amount from about 1% to about 50% by weight;

PEO in an amount from about 1% to about 60% by weight;
HPMC in an amount from about 1% to about 60% by weight;
Lactose in an amount from about 0% to about 80% by weight;
Microcrystalline Cellulose in an amount from about 1% to about 99% by weight;
and magnesium stearate in an amount from about 0.05% to about 10% by weight.

The carvedilol can be present in an amount selected from about 0.1%, about 0.5%, about 1%, about 5%, about 10%, about 15%, about 20%, about 25%, about 30%, about 35%, about 40%, about 45% or about 50% by weight.

The Eudragit® E can be present in an amount selected from about 1%, about 5%, about 10%, about 15%, about 20%, about 25%, about 30%, about 35%, about 40%, about 45% or about 50% by weight.

The PEO can be present in an amount selected from about 1%, about 5%, about 10%, about 15%, about 20%, about 25%, about 30%, about 35%, about 40%, about 45%, about 50%, about 55% or about 60% by weight.

The HPMC can be present in an amount selected from about 1%, about 5%, about 10%, about 15%, about 20%, about 25%, about 30%, about 35%, about 40%, about 45%, about 50%, about 55% or about 60% by weight.

The lactose can be present in an amount selected from about 0%, about 5%, about 10%, about 15%, about 20%, about 25%, about 30%, about 35%, about 40%, about 45%, about 50%, about 55%, about 60%, about 65%, about 70%, about 75% or about 80% by weight.

The microcrystalline cellulose can be present in an amount selected from about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 99% by weight.

The magnesium stearate can be present in an amount selected from about 0.05%, about 0.1%, about 1%, about 5% or about 10% by weight.

In a further aspect a controlled release dosage form is provided wherein said dosage form comprises

carvedilol in an amount of about 7.7% by weight;
Eudragit® E in an amount of about 11.5% by weight;
PEO in an amount of about 10% by weight;
HPMC in an amount of about 10% by weight;
Lactose in an amount of about 29.9% by weight;
Microcrystalline Cellulose in an amount of about 29.9% by weight;
and magnesium stearate in an amount of about 1% by weight.

In another aspect the invention provides a controlled release dosage form comprising a tablet comprising

| | % W/W |
|---|---|
| Carvedilol | 0.96 |
| PEO | 10.00 |
| HPMC | 10.00 |
| HO(C₂H₄O)₁₀₁(C₃H₆O)₅₆(C₂H₄O)₁₀₁H | 20.00 |
| (Pluronic ® F127) | |
| Microcrystalline Cellulose | 28.96 |
| Lactose Monohydrate | 28.97 |
| Polyvinyl alcohol | 0.11 |

### Magnesium Stearate 1.00

wherein the dosage form is adapted to release the carvedilol into an acidic medium at the following rates measured using the method of United States Pharmacopoeia No. II at 150 rpm in 900 ml pH 4.5 acetate buffer:
about 9% after about 0.5 hours;
about 26% after about 2 hours;
about 46% after about 4 hours;
about 82% after about 8 hours;
about 99% after about 12 hours and
about 100% after about 16 hours.

In a further aspect the invention provides a controlled release dosage form comprising a tablet comprising

| | % W/W |
|---|---|
| Carvedilol | 1.92 |
| PEO | 10.00 |
| HPMC | 10.00 |
| HO(C₂H₄O)₁₀₁(C₃H₆O)₅₆(C₂H₄O)₁₀₁H (Pluronic ® F127) | 20.00 |
| Microcrystalline Cellulose | 28.43 |
| Lactose Monohydrate | 28.43 |
| Polyvinyl alcohol | 0.22 |
| Magnesium Stearate | 1.00 |

wherein the dosage form is adapted to release the carvedilol into an acidic medium at the following rates measured using the method of United States Pharmacopoeia No. II at 150 rpm in 900 ml pH 4.5 acetate buffer:
about 7% after about 0.5 hours;
about 22% after about 2 hours;
about 44% after about 4 hours;
80% after about 8 hours; and
100% after about 12 hours.

In a still further aspect the invention provides a controlled release dosage form comprising a tablet comprising

| | % W/W |
|---|---|
| Carvedilol | 3.85 |
| PEO | 10.00 |
| HPMC | 10.00 |
| HO(C₂H₄O)₁₀₁(C₃H₆O)₅₆(C₂H₄O)₁₀₁H (Pluronic ® F127) | 20.00 |
| Microcrystalline Cellulose | 27.26 |
| Lactose Monohydrate | 27.46 |
| Polyvinyl alcohol | 0.44 |
| Magnesium Stearate | 1.00 |

wherein the dosage form is adapted to release the carvedilol into an acidic medium at the following rates measured using the method of United States Pharmacopoeia No. II at 150 rpm in 900 ml pH 4.5 acetate buffer:
about 7% after about 0.5 hours;
about 23% after about 2 hours;
about 43% after about 4 hours;
about 79% after about 8 hours; and
about 100% after about 12 hours.

In another aspect of the invention a controlled release dosage form is provided wherein said dosage form comprises a tablet comprising

| | % W/W |
|---|---|
| Carvedilol | 7.69 |
| PEO | 10.00 |
| HPMC | 10.00 |
| HO(C₂H₄O)₁₀₁(C₃H₆O)₅₆(C₂H₄O)₁₀₁H (Pluronic ® F127) | 20.00 |
| Microcrystalline Cellulose | 25.52 |
| Lactose Monohydrate | 25.53 |
| Polyvinyl alcohol | 0.26 |
| Magnesium Stearate | 1.00 |

wherein the dosage form is adapted to release the carvedilol into an acidic medium at the following rates measured using the method of United States Pharmacopoeia No. II at 150 rpm in 900 ml pH 4.5 acetate buffer:
about 9% after about 0.5 hours;
about 23% after about 2 hours;
about 42% after about 4 hours;
about 78% after about 8 hours; and
about 100% after about 12 hours.

In another aspect the invention provides a controlled release dosage form comprising a tablet comprising

| | % W/W |
|---|---|
| Carvedilol | 11.54 |
| PEO | 10.00 |
| HPMC | 10.00 |
| HO(C₂H₄O)₁₀₁(C₃H₆O)₅₆(C₂H₄O)₁₀₁H (Pluronic ® F127) | 20.00 |
| Microcrystalline Cellulose | 23.54 |
| Lactose Monohydrate | 23.53 |
| Polyvinyl alcohol | 0.39 |
| Magnesium Stearate | 1.00 |

wherein the dosage form is adapted to release the carvedilol into an acidic medium at the following rates measured using the method of United States Pharmacopoeia No. II at 150 rpm in 900 ml pH 4.5 acetate buffer:
about 8% after about 0.5 hours;
about 21% after about 2 hours;
about 39% after about 4 hours;
about 75% after about 8 hours; and
about 100% after 12 hours.

In another aspect the invention provides a controlled release dosage form comprising a tablet comprising

| | % W/W |
|---|---|
| Carvedilol | 14.29 |
| PEO | 10.00 |
| HPMC | 10.00 |
| HO(C₂H₄O)₁₀₁(C₃H₆O)₅₆(C₂H₄O)₁₀₁H (Pluronic ® F127) | 20.00 |
| Microcrystalline Cellulose | 22.12 |
| Lactose Monohydrate | 22.11 |
| Polyvinyl alcohol | 0.48 |
| Magnesium Stearate | 1.00 |

wherein the dosage form is adapted to release the carvedilol into an acidic medium at the following rates measured using the method of United States Pharmacopoeia No. II at 150 rpm in 900 ml pH 4.5 acetate buffer:
about 7% after about 0.5 hours;
about 20% after about 2 hours;
about 37% after about 4 hours;
about 70% after about 8 hours;
about 100% after 12 hours.

In a further aspect the invention provides a controlled release dosage form comprising a tablet comprising:

| | % W/W |
|---|---|
| Carvedilol | 7.7 |
| PEO WSR Coagulant | 10 |
| Methocel K4M | 10 |
| HO(C₂H₄O)₁₀₁(C₃H₆O)₅₆(C₂H₄O)₁₀₁H (Pluronic ® F127) | 20 |
| Microcrystalline cellulose | 25.65 |
| Lactose monohydrate | 25.65 |
| Magnesium Stearate | 1 |

wherein said controlled release tablet when administered to a subject in need of such administration, following a fast of at least 4 hours, with 240 ml of water 30 minutes after the start of an American Heart Association meal, exhibits a mean AUC(0-inf) for carvedilol of about 588.67 ng*hr/ml after administration of a once daily 50 mg dose of said controlled release carvedilol tablet. The controlled release dosage form according to this aspect of the invention exhibits a mean AUC(0-t) for carvedilol of about 583.96 ng*hr/ml after administration of a once-daily 50 mg dose of said controlled release carvedilol tablet. The controlled release dosage form according to this aspect of the invention exhibits a mean Cmax for carvedilol of about 61.52 ng/ml after administration of a once-daily 50 mg dose of said controlled release carvedilol tablet. The controlled release dosage form according to this aspect of the invention exhibits a median Tmax for carvedilol of about 6.85 hr after administration of a once-daily 50 mg dose of said controlled release carvedilol tablet.

In a further aspect the invention provides a controlled release dosage form comprising a tablet comprising:

| | % W/W |
|---|---|
| Carvedilol | 7.7 |
| PEO WSR Coagulant | 10 |
| Methocel K100M | 10 |
| HO(C₂H₄O)₁₀₁(C₃H₆O)₅₆(C₂H₄O)₁₀₁H (Pluronic ® F127) | 20 |
| Microcrystalline cellulose | 25.65 |
| Lactose monohydrate | 25.65 |
| Magnesium Stearate | 1 |

wherein said controlled release tablet when administered to a subject in need of such administration, following a fast of at least 4 hours, with 240 ml of water 30 minutes after the start of an American Heart Association meal, exhibits a mean AUC(0-inf) for carvedilol of about 571.56 ng*hr/ml after administration of a once daily 50 mg dose of said controlled release carvedilol tablet. The controlled release dosage form according this aspect of the invention exhibits a mean AUC(0-t) for carvedilol of about 550.7 ng*hr/ml after administration of a once-daily 50 mg dose of said controlled release carvedilol tablet. The controlled release dosage form according to this aspect of the invention exhibits a mean Cmax for carvedilol of about 58.82 ng/ml after administration of a once-daily 50 mg dose of said controlled release carvedilol tablet. The controlled release dosage form according to this aspect of the invention exhibits a median Tmax for carvedilol of about 8.25 hr after administration of a once-daily 50 mg dose of said controlled release carvedilol tablet.

In another aspect the invention provides a controlled release dosage form comprising a tablet comprising:

| | % W/W |
|---|---|
| Carvedilol/methyl methacrylate 1:2:1 (Eudragit ® E) 40:60 | 19.25 |
| extrudate | |
| PEO WSR Coagulant | 10.0 |
| Methocel K4M | 10.0 |
| Microcrystalline cellulose | 29.9 |
| Lactose monohydrate | 29.9 |
| Magnesium Stearate | 1 |

wherein said controlled release tablet when administered to a subject in need of such administration, following a fast of at least 4 hours, with 240 ml of water 30 minutes after the start of an American Heart Association meal, exhibits a mean AUC(0-inf) for carvedilol of about 593.54 ng*hr/ml after administration of a once daily 50 mg dose of said controlled release carvedilol tablet. The controlled release dosage form according to this aspect of the invention exhibits a mean AUC(0-t) for carvedilol of about 584.33 ng*hr/ml after administration of a once-daily 50 mg dose of said controlled release carvedilol tablet. The controlled release dosage form according to this aspect of the invention exhibits a mean Cmax for carvedilol of about 70.84 ng/ml after administration of a once-daily 50 mg dose of said controlled release carvedilol tablet. The controlled release dosage form according to this aspect of the invention exhibits a median Tmax for carvedilol of about 6.5 hr after administration of a once-daily 50 mg dose of said controlled release carvedilol tablet.

In a further aspect the invention provides a controlled release dosage form comprising a tablet comprising:

| | % W/W |
|---|---|
| Carvedilol/methyl methacrylate 1:2:1 (Eudragit ® E) 30:70 | 25.6 |
| extrudate | |
| PEO WSR Coagulant | 10.0 |
| Methocel K100M | 10.0 |
| Microcrystalline cellulose | 53.4 |
| Magnesium Stearate | 1 |

wherein said controlled release tablet when administered to a subject in need of such administration, following a fast of at least 4 hours, with 240 ml of water 30 minutes after the start of an American Heart Association meal, exhibits a mean AUC(0-inf) for carvedilol of about 613.75 ng*hr/ml after administration of a once daily 50 mg dose of said controlled release carvedilol tablet. The controlled release dosage form according to this aspect of the invention exhibits a mean AUC(0-t) for carvedilol of about 595.78 ng*hr/ml after administration of a once-daily 50 mg dose of said controlled release carvedilol tablet. The controlled release dosage form according to this aspect of the invention exhibits a mean Cmax for carvedilol of about 80.01 ng/ml after administration of a once-daily 50 mg dose of said controlled release carvedilol tablet. The controlled release dosage form according to this aspect of the invention exhibits a median Tmax for carvedilol of about 8.45 hr after administration of a once-daily 50 mg dose of said controlled release carvedilol tablet.

The present invention also provides a method of treating a cardiovascular condition in a subject in need of such treatment comprising administering to said subject a controlled release dosage form according to the present invention. The dosage form according to the invention can be used to treat a cardiovascular condition selected from the group consisting of hypertension, congestive heart failure and left ventricular dysfunction following myocardial infarction. The dosage form of the invention can be administered once daily for treating a cardiovascular condition in a subject in need of such treatment.

The dosage forms according to the present invention can further comprise a coat. The coat can comprise a cosmetic overcoat. For example the coat can be a coloured coat. The colour of coat can be selected in order to differentiate between different strength tablets.

In an alternative embodiment the dosage form can comprise a coat comprising a second drug. The second drug can be selected from the group consisting of ACE inhibitors, diuretics, and digoxin. In one aspect of the invention, the ACE inhibitors can be selected from the group consisting of captopril, lisinopril, ramipril and enalapril, or any pharmaceutically acceptable salts thereof. The diuretics can comprise hydrochlorothiazide or furosemide, or any pharmaceutically acceptable salts thereof. Such dosage forms would be suitable for use for example in combination applications.

### BRIEF DESCRIPTION OF THE DRAWINGS

The invention will be described in more detail with reference to the accompanying drawings in which

FIG. 1 shows the dissolution profile for each of the formulations set out in Table 1;

FIG. 2 shows the dissolution profile for each of the formulations set out in Table 2;

FIG. 3 shows the dissolution profile for each of the formulations set out in Table 3;

FIG. 4 shows the dissolution profile for each of the formulations set out in Table 6;

FIG. 5 shows the mean plasma carvedilol concentrations (n=20) for Carvedilol 50 mg ER tablets q.d. (once-daily) v's Coreg 25 mg b.i.d.;

FIG. 6 shows the dissolution profile for the formulations set out in Table 10;

FIG. 7 shows the dissolution profile for the formulations set out in Table 11;

FIG. 8 shows the dissolution profile for the formulations set out in Table 12;

FIG. 9 shows the dissolution profiled for the formulations set out in Table 14; and

FIG. 10 shows the mean plasma carvedilol concentrations (n=20) for carvedilol 50 mg ER tablets q.d. (once-daily) v's Coreg 25 mg b.i.d.

### DEFINITIONS

The term “active”, “active agent”, “active pharmaceutical agent”, “active drug” or “drug” as used herein means any active pharmaceutical ingredient (“API”), including its pharmaceutically acceptable salts, as well as in the anhydrous, hydrated, and solvated forms, in the form of prodrugs, and in the individually optically active enantiomers of the API as well as polymorphs of the API.

The term “other drug” or “second drug” as used herein means a drug other than carvedilol, including but not limited to anti-depression agents, other neuropsychiatric drugs, vasodilators, anti-anxiety agents, appetite modulators, sleep modulating drugs, SSRIs, anti-viral agents, anti-pain agents, anti-migraine agents, anti-inflammatories (both steroidal and non-steroidal) and more particularly can include citalopram, escitalopram, venlafaxine, clozapine, melperone, amperozide, iloperidone, risperidone, quetiapene, olanzapine, ziprasidone, aripiprazole, reboxetine, Viagra®, sertraline, paroxetine, fluoxetine, gabapentin, valproic acid, amitriptyline, lofepramine, fluvoxamine, imipramine, mirtazapine, nefazodone, nortriptyline, S-adenosylmethionine (SAM-E), combinations thereof, and their pharmaceutically acceptable salts (e.g. the hydrochloride salts, the hydrobromide salts, the hydroiodide salts, and the saccharinate salts), as well as in the anhydrous, hydrated, and solvated forms, in the form of prodrugs, and in the individually optically active enantiomers of the drug, ACE inhibitors, diuretics, and digoxin.

The term “formulation” or “composition” as used herein refers to the drug in combination with pharmaceutically acceptable carriers and additional inert ingredients. This includes orally administrable formulations as well as formulations administrable by other means.

The term “dosage form” as used herein is defined to mean a pharmaceutical preparation in which doses of active drug are included.

“Modified release dosage forms” as used herein is as defined by the United States Pharmacopoeia (USP) as those whose drug release characteristics of time course and/or location are chosen to accomplish therapeutic or convenience objectives not offered by conventional, immediate release or uncoated normal matrix dosage forms. The rate of release of the active drug from a modified release dosage form is controlled by features of the dosage form and/or in combination with physiologic or environmental conditions rather than by physiologic or environmental conditions alone. The modified release dosage forms of the invention can be contrasted to conventional, immediate release, or uncoated normal matrix dosage forms which forms typically produce large maximum/minimum plasma drug concentrations (Cmax/Cmin) due to rapid absorption of the drug into the body (i.e., in vivo, relative to the drug's therapeutic index; i.e., the ratio of the maximum drug concentration needed to produce and maintain a desirable pharmacological response). In conventional, immediate release or uncoated normal matrix dosage forms, the drug content is released into the gastrointestinal tract within a short period of time, and plasma drug levels peak shortly after dosing. The design of conventional, immediate release or uncoated normal matrix dosage forms is generally based on getting the fastest possible rate of drug release, and therefore absorbed, often at the risk of creating undesirable dose related side effects. The modified release dosage forms of the invention, on the other hand, improve the therapeutic value of the active drug by reducing the ratio of the maximum/minimum plasma drug concentration (Cmax/Cmin) while maintaining drug plasma levels within the therapeutic window. The modified release dosage forms of the invention attempt to deliver a therapeutically effective amount of carvedilol as a once-daily dose so that the ratio Cmax/Cmin in the plasma at steady state is less than the therapeutic index, and to maintain drug levels at constant effective levels to provide a therapeutic benefit over a 24-hour period. The modified release dosage forms of the invention, therefore, avoid large peak-to-trough fluctuations normally seen with conventional or immediate release dosage forms and can provide a substantially flat serum concentration curve throughout the therapeutic period. Modified release dosage forms can be designed to provide a quick increase in the plasma concentration of carvedilol which remains substantially constant within the therapeutic range of carvedilol for at least a 24-hour period. Alternatively, modified-release dosage forms can be designed to provide a quick increase in the plasma concentration of carvedilol, which although can not remain constant, declines at rate such that the plasma concentration remains within the therapeutic range for at least a 12 hour and desirably at least a 24-hour period.

The modified release dosage forms of the invention can be constructed in many forms known to one of ordinary skill in the drug delivery arts and described in the prior art such as for example, “modified release matrix dosage forms”, “osmotic dosage forms”, “multiparticulate dosage forms”, and “gastric retention dosage forms”.

The USP considers that the terms controlled release, prolonged release and sustained release are interchangeable. Accordingly, the terms “modified-release”, controlled-release”, “control-releasing”, “rate-controlled release”, “prolonged-release”, and “sustained-release” are used interchangeably herein. For the discussion herein, the definition of the term “modified-release” encompasses the scope of the definitions for the terms “extended release”, “enhanced-absorption”, “controlled release”, and “delayed release”.

“Controlled release dosage forms” or “control-releasing dosage forms”, or dosage forms which exhibit a “controlled release” of carvedilol as used herein is defined to mean dosage forms administered once-daily that release the carvedilol at a controlled rate and provide plasma concentrations of the carvedilol that remain controlled with time within the therapeutic range of the carvedilol over a 24-hour period. “Controlled release” or “control releasing” is defined to mean release of the drug gradually or in a controlled manner per unit time. For example, the controlled rate can be a constant rate providing plasma concentrations of the carvedilol that remain invariant with time within the therapeutic range of carvedilol over at least a 12 or 24-hour period.

“Sustained-release dosage forms” or dosage forms which exhibit a “sustained-release” of carvedilol as used herein is defined to mean dosage forms administered once-daily that provide a release of the carvedilol sufficient to provide a therapeutic dose soon after administration, and then a gradual release over an extended period of time such that the sustained-release dosage form provides therapeutic benefit over a 12 or 24-hour period.

“Extended- or sustained-release dosage forms” or dosage forms which exhibit an “extended or sustained release” of carvedilol as used herein is defined to include dosage forms administered once-daily that release the carvedilol slowly, so that plasma concentrations of carvedilol are maintained at a therapeutic level for an extended period of time such that the extended or sustained-release dosage form provides therapeutic benefit over a 12 or 24-hour period.

“Enhanced absorption dosage forms” or dosage forms which exhibit an “enhanced absorption” of the carvedilol is defined to mean dosage forms that when exposed to like conditions, will show higher release and/or more absorption of the carvedilol base as compared to other dosage forms with the same or higher amount of carvedilol base. The same therapeutic effect can be achieved with less carvedilol base in the enhanced absorption dosage form as compared to other dosage forms.

The term “controlled release matrix” is defined to mean a dosage form in which the carvedilol is dispersed within a matrix, which matrix can be either insoluble, soluble, or a combination thereof. Controlled release matrix dosage forms of the insoluble type are also referred to as “insoluble polymer matrices”, “swellable matrices”, or “lipid matrices” depending on the components that make up the matrix. Controlled release matrix dosage forms of the soluble type are also referred to as “hydrophilic colloid matrices” or “erodible matrices”. Controlled release matrix dosage forms of the invention refer to dosage forms comprising an insoluble matrix, a soluble matrix or a combination of insoluble and soluble matrices in which the rate of release is slower than that of an uncoated non-matrix conventional or immediate release dosage forms or uncoated “normal release matrix” dosage forms. Controlled release matrix dosage forms can be coated with a “control-releasing coat” to further slow the release of carvedilol from the controlled release matrix dosage form. Such coated controlled release matrix dosage forms can exhibit “modified-release”, controlled-release”, “sustained-release”, “extended-release”, “prolonged release”, “delayed-release” or combinations thereof of carvedilol.

The term “normal release matrix” is defined to mean dosage forms in which carvedilol is dispersed within a matrix, which matrix can be either insoluble, soluble, or combinations thereof but constructed such that the release of the carvedilol mimics the release rate of an uncoated non-matrix conventional or immediate release dosage form comprising carvedilol. The release rate from normal release matrix dosage forms can be slowed down or modified in conjunction with a “control releasing coat”.

The term “gastric retention delivery system” as used herein means a drug delivery system or dosage form that is designed to prolong the residence time of the active agent and dosage form in the stomach. In a gastrically retained formulation the dosage form is small enough to be swallowed comfortably but swells to a size upon contact with the gastric fluid in the stomach such that it is retained in the stomach.

The term “solid dispersion” as used herein means an apparently homogeneous solid substance which consists of a microscopically heterogenous mixture of the active agent and the extrusion polymer. A solid dispersion according to the invention can include the active agent in a range of physical states ranging from molecular dispersion to amorphous or pre-crystalline associations of molecules to nanoparticulate domains.

The term “extrusion polymer” as used herein refers to a polymeric material capable of forming a solid dispersion of amorphous drug substance when both extrusion polymer and drug substance are co-processed

The term “bio-available” means a condition which permits the active ingredient to interact with, i.e. become available for use in, the target bio-system, i.e. the body of the host animal or human patient.

The term “medicament” as used herein refers to all possible oral and non-oral dosage forms, including but not limited to, all modified release dosage forms, osmosis controlled release systems, erosion controlled release systems, dissolution controlled release systems, diffusion controlled release systems, matrix tablets, enteric coated tablets, single and double coated tablets (including the extended release and enhanced absorption tablets as described herein), capsules, minitablets, caplets, coated beads, granules, spheroids, pellets, microparticles, suspensions, topicals such as transdermal and transmucoasal compositions and delivery systems (containing or not containing matrices), injectables, and inhalable compositions.

The term “core” as used herein is defined to mean any structure that is surrounded by a wall, membrane, or coating. The wall, membrane, or coating can be a functional or non-functional coating.

The term “tablet” as used herein refers to a single dosage form, i.e. the single entity containing the active pharmaceutical agent that is administered to the subject. The term “tablet” also includes a tablet that can be the combination of one or more “minitablets”.

The term “pharmaceutically acceptable” means compatible with the treatment of subjects, in particular, humans.

The term “subject” or “patient” as used herein means all members of the animal kingdom, in particular, humans.

The term “effective amount” as used herein means a “pharmaceutically effective amount”.

A “pharmaceutically effective amount” is the amount or quantity of the carvedilol or polymorph or enantiomer thereof which is sufficient to elicit an appreciable biological response when administered to a patient. It will be appreciated that the precise therapeutic dose will depend on the age and condition of the patient and the nature of the condition to be treated and will be at the ultimate discretion of the attendant physician.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (ie. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “a” or “an” as used herein means “one” or “one or more”.

Unless indicated to the contrary, the numerical parameters set forth in the following specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Other terms are defined as they appear in the following description and should be construed in the context with which they appear.

### DETAILED DESCRIPTION OF THE INVENTION

In one aspect, the invention provides a controlled (extended) release dosage form of carvedilol which is suitable for gastric retention. Carvedilol has very low aqueous solubility. The solubility of Carvedilol at pH 7 is 23 g/ml and at pH 5 is 100 μg/ml. Low solubility drugs such as carvedilol can possess poor relative bioavailability in the distal small intestine and large intestine.

The present invention addresses the limitation of swellable, gastrically retained tablets for delivery of poorly soluble drugs, such as carvedilol, to the proximal small intestine. In particular the present invention addresses the significant bioavailability challenges that exist when carvedilol is released from a sustained release formulation in the distal small intestine or large intestine.

In general, for matrix systems employing hydrophilic swellable polymers to control rate of drug release, the higher the molecular weight or viscosity of a rate controlling polymer, the greater the barrier to drug diffusion and therefore the slower the rate of drug release. For poorly water soluble drugs, relatively low molecular weight/viscosity polymers are generally employed to facilitate drug release through erosion of the matrix (Skoug et al (1993), Journal of Controlled Release 27, 227-245; Tahara et al (1995) Journal of Controlled Release 35, 59-66). Conversely, for soluble drugs, high molecular weight polymers are employed which swell, thus creating a situation where drug diffusion contributes significantly to the rate of drug release.

Given the low solubility of carvedilol it would be preferable to formulate matrix tablets for controlled release using relatively low molecular weight polymers, in which drug release is controlled over time by matrix erosion. This approach is not ideal for gastrically retained systems where the dosage form is intended to be retained for a prolonged period in the stomach to maximize drug delivery to the proximal small intestine, as tablet erosion will inevitably lead to premature loss of the dosage form through the pyloric sphincter as its size reduces. It would be preferable to use high molecular weight polymers that will swell and withstand the mechanical stress in the stomach, thus retaining its physical integrity during the process of drug release. It is highly likely that in such cases however, complete drug release will not occur in the stomach or proximal small intestine given the diffusion barrier to drug release which would be created by the high molecular weight/viscosity polymers.

U.S. Pat. No. 6,723,340 teaches that in order to overcome the challenge of delivering a sustained release dose of a poorly soluble drug from a gastric retention system based upon hydrophilic polymers, the molecular weight and viscosity of the polymers should be reduced to encourage an erosion based release. However, such an approach has the potential to suffer from the disadvantage of either (i) premature drug release (dose dumping) due to the use of polymers of insufficient gel strength in the hydrated state to withstand the shear forces present in the stomach in the fed state and/or (ii) insufficient swelling and therefore insufficient gastric retention.

The use of low viscosity polymers is undesirable for gastrically retained formulations because the rate and extent of swelling of such polymers together with the rate of erosion, will not enable the size of dosage form and long-lasting integrity required to maintain gastric retention.

Accordingly, in one embodiment of the invention, the dosage form described herein comprises a hydrophilic matrix tablet with sufficient swelling properties to ensure retention of the dosage form in the stomach for as long as possible. The dosage form according to the invention also possesses sufficient erosion properties to ensure adequate rate and extent of drug release in both the stomach and small intestine.

The dosage form described herein can comprise high molecular weight and/or high viscosity hydrophilic matrix polymers. In order to achieve an effective balance between swelling and erosion of the dosage form, the hydrophilic polymers Hydroxypropylmethylcellulose (HPMC) and Polyethyleneoxide (PEO) offer the potential to optimize a gastric retention extended release carvedilol formulation.

The use of such high molecular weight and/or high viscosity hydrophilic matrix polymers presents a significant diffusion barrier for poorly soluble drugs, such as carvedilol, thereby preventing adequate drug release. Accordingly, the dosage form according to the present invention further comprises additional functional excipients in order to influence the release of low solubility drugs. The incorporation of additional functional excipients alleviates the need to compromise swelling and the integrity of the dosage form by reducing the hydrophilic polymer molecular weight and/or viscosity.

Accordingly, in one aspect of the present invention, polyalkylene block copolymers (such as Pluronic®s (also known as Poloxamers or Lutrols) are included in the dosage form as drug release modifiers. Unlike in U.S. Pat. No. 5,393,765 where polyalkylene block copolymers are considered erosion enhancers, it has surprisingly been found that the polyalkylene block copolymers influence drug release by a mechanism based on a mechanism other than erosion alone.

The data presented in the examples for the dosage forms according to the present invention demonstrates that a formulation in which the polyalkylene block copolymer is substituted with an alternative excipient such as lactose or microcrystalline cellulose does not facilitate the same rate and extent of drug release as a formulation comprising Poloxamer. Without being bound by any theory, it is considered that the polyalkylene block copolymer operates as both an erosion enhancer and solubility enhancer, thereby enabling greater extent of drug release from a formulation comprising high molecular weight and viscosity PEO and HPMC respectively.

Additionally data presented herein (Tables 6, 8 and 9) demonstrates that formulations of the invention comprising high molecular weight PEO and high viscosity HPMC in combination with polyoxyalkylene copolymers are more effective in delivering a sustained release dose of carvedilol than a formulation with a lower molecular weight PEO in the absence of a polyoxyalkylene copolymer.

In a further aspect of the present invention the dosage form comprises carvedilol stabilised in its amorphous form through the formation of a solid dispersion with a polymer such as Eudragit® E in which the solubility of the drug is significantly enhanced. As a result, when incorporated into a matrix of high molecular weight hydrophilic polymers, the improved solubility of the drug enables drug diffusion to occur despite the significant dissolution barrier created by the high molecular weight hydrophilic polymers. As a result, a dosage form for the controlled release of carvedilol can be designed that withstands the mechanical stress of the stomach during the fed state, thus maintaining its physical integrity and extending its retention in the stomach lumen for as long as possible, whilst enabling drug release via drug diffusion to occur.

While the present invention is directed towards a dosage form of carvedilol which is suitable for gastric retention, the skilled person will appreciate that the dosage form described herein can be used for the delivery of other low solubility drugs. In particular the skilled person will appreciate that the gastrically retained dosage form according to the invention can be used for the delivery of drugs having a solubility of below approximately 0.5 parts drug in 10 parts water, in particular below about 0.3 parts per 10 parts water.

### EXAMPLES

In order that the present invention can be more readily understood, the following examples are given, by way of illustration only. They are not in any way intended to limit the scope of the protection provided herein.

For examples where dissolution data is provided the dissolution method used is that set out for Example 1 except where otherwise stated.

In all instances where prophetic examples are provided these compositions are intended to be exemplary and it should be understood that the specific procedures, constituents, amounts thereof and the like can be varied in order to obtain a composition possessing desired properties.

The dosage forms according to the invention wherein the means for enhancing the rate and/or extent of release of carvedilol comprises a polyoxyalkylene block co-polymer can be prepared as described below.

In one embodiment, tablets of the invention can be prepared, for example, by direction compression of carvedilol and excipients. The skilled person will appreciate that there is an array of different ways in which a tablet blend can be produced that is suitable for compression into tablets with acceptable drug content uniformity.

In one embodiment, for the manufacture of a 4 kg batch of 50 mg carvedilol tablets, half the required amount of microcrystalline cellulose, half the required amount of lactose, half the required amount of PEO, half the required amount of HPMC and half the required amount of Pluronic® are filled into a Pharmatech AB-050 V Shell blender. Subsequently, the carvedilol, with the remaining microcrystalline cellulose, lactose, PEO, HPMC and Pluronic® are added to the Blender. The blend is then mixed at 25 rpm for 10 minutes without the use of an intensifier bar. Following the 10 minutes blending, the magnesium stearate is added to the blend, and the blend further tumbled in the V Blender for one minute at 25 rpm without the use of the intensifier. The tablet blend is discharged from the V Blender and compressed into tablets using a Riva Picolla Rotary tablet press model B/10 fitted with 17 mm×9 mm caplet tooling. Compression parameters are adjusted in order to achieve a tablet weight of 650 mg and hardness of 80-120N.

In an alternative embodiment, carvedilol is granulated prior to mixing with other tablet excipients, in order to improve powder slow during compression. Granulation can be achieved through either wet or dry granulation. In one embodiment of the invention, in order to manufacture a 30 kg batch of 50 mg carvedilol tablets, carvedilol is first wet granulated with lactose and polyvinyl alcohol (PVA) as a binder in an Aeromatic Fielder MP3/2/3 fluidized bed granulator. In brief, the granulation binder solution is prepared by dispersing the PVA in cold water which is subsequently heated to approximately 60° C. to solubilize the PVA. The solution is then allowed to cool for at least 2 hours. The granulation solution is then top-sprayed onto a 18 kg fluidized bed of carvedilol and lactose (58.41:41.59 ratio of lactose:carvedilol), fluidized in a Aeromatic Fielder MP3/2/3 fluidized bed granulator with the following process conditions:

| Process Parameter | Setting | |
|---|---|---|
| Product Temperature | 25-26° | C. |
| Inlet Air Temperature | 65° | C. |
| Air velocity | 250 | m³/h |
| Atomising Air Pressure | 1 | bar |
| Spray Rate | 70 | g/min |

Following application of 252 g of PVA to the fluidized bed, spraying is stopped and the granules further fluidized to dry the granulates to a moisture content of approximately 1.5% w/w.

To blend the carvedilol granules with the other tablet excipients, half the required amount of microcrystalline cellulose, half the required amount of lactose, half the required amount of PEO, half the required amount of HPMC and half the required amount of the Pluronic® are filled into a Pharmatech AB-400 V Shell blender. Subsequently, the carvedilol granules, with the remaining microcrystalline cellulose, lactose, PEO, HPMC and Pluronic® are added to the Blender. The 30 kg blend is then mixed at 25 rpm for 10 minutes without the use of an intensifier bar. Following the 10 minutes blending, the magnesium stearate is added to the blend, and the blend further tumbled in the V Blender for one minute at 25 rpm without the use of the intensifier. The tablet blend is discharged from the V Blender and compressed into tablets using a Fette 1200 tablet press fitted with 17 mm×9 mm caplet tooling. Compression parameters are adjusted in order to achieve a tablet weight of 650 mg and hardness of 80-120N.

The dosage forms according to the invention wherein the means for enhancing the rate and/or extent of release of carvedilol comprises a solid dispersion of carvedilol and an extrusion polymer can be prepared as described below.

In one embodiment of the invention a solid dispersion of carvedilol is formed prior to its inclusion into the tablet blend. In one embodiment, the solid dispersion of the invention is prepared by a solvent evaporation method, in which carvedilol and a carrier are dissolved in a common solvent which is subsequently removed by evaporation under vacuum, spray drying or freeze drying to produce the solid dispersion, also known as a co-precipitation. Given the environmental concerns over the excessive use of solvents, a preferred method of manufacture of the solid dispersion is through the use of hot melt extrusion in which both carvedilol and a carrier are heated above their melting point and the molten mix processed with for example a twin-screw extruder, milled and rapidly cooled to form the solid dispersion. Hot melt spinning is an alternative way of producing solid dispersion, in which carvedilol and carrier are melted together over an extremely short time in a high speed mixer of centrifugal apparatus and the extruded material dispersed in air or an inert gas in a cooling tower.

By way of example, the process for manufacture of a 30:70 carvedilol:Eudragit® E extrudate is as follows. Each heating zone of an APV Baker 19 mm twin-screw extruder is heated to a target temperature of 70° C., 140° C., 140° C., 130° C., and 100° C. for each of heating zones 1, 2, 3, 4 and 5 respectively. The extruder twin screws are then rotated at 140 rpm and a 4.6 kg blend of carvedilol and Eudragit® E, preblended in a Pharmatech AB50 V blender for 5 minutes, fed into the extruder hopper until all five heating zone temperatures are within 5° C. of the target temperature. Extrusion of the blend is continued at 140 rpm and milled extrudate is collected on a stainless steel tray.

In order to manufacture a 4 kg batch of 50 mg carvedilol tablets comprising the melt extrusion, half the required amount of microcrystalline cellulose, half the required amount of lactose, half the required amount of PEO, half the required amount of HPMC and half the required amount of Pluronic® are filled into a Pharmatech AB-050 V Shell blender. Subsequently, the carvedilol extrudate, with the remaining microcrystalline cellulose, lactose, PEO, HPMC and Pluronic® are added to the Blender. The blend is then mixed at 25 rpm for 10 minutes without the use of an intensifier bar. Following the 10 minutes blending, the magnesium stearate is added to the blend, and the blend further tumbled in the V Blender for one minute at 25 rpm without the use of the intensifier. The tablet blend is discharged from the V Blender and compressed into tablets using a Riva Picolla Rotary tablet press model B/10 fitted with 17 mm×9 mm caplet tooling. Compression parameters are adjusted in order to achieve a tablet weight of 650 mg and hardness of 80-120N.

### Example 1

A comparison of the following formulations demonstrates the invention.

EO956 is a 650 mg 17 mm×9 mm tablet matrix formulation (hardness 60-80N) comprising 50 mg carvedilol, 10% w/w 5,000,000 MW Polyethylene oxide (PEO WSR Coag.), 10% w/w 4,000 cps HPMC (Methocel K4M) together with 20% polyoxyalkylene block copolymer (Pluronic® F127) as a drug release modifier.

EO939 is a tablet identical in size and shape and hardness to EO956, has the same levels of K4M and PEO WSR Coag., but differs in that the Pluronic® F127 is replaced with lactose as a drug release modifier.

EO929 is a tablet identical in size and shape and hardness to EO956, has the same levels of Methocel K4M and PEO WSR Coag., but differs from both EO956 and EO939 in that both Pluronic® F127 and lactose are present in the formulation.

### Dissolution Method:

Dissolution was performed in a US Pharmacopeia 27 dissolution apparatus II (paddles). Given the swellable and potentially floatable nature of the carvedilol tablets, tablets were loosely wrapped in copper wire (<165 mm×0.5 mm) to hold them in place without restricting swelling. Dissolution was performed at 75 rpm in 900 ml of 0.1N HCl+5% Tween.

TABLE 1

| | Carvedilol example | | |
|---|---|---|---|
| | formulations | | |
| Components of Tablet | EO956 | EO939 | EO929 |
| Formulation (%) | (%) | (%) | (%) |
| Carvedilol | 7.7 | 7.7 | 7.7 |
| PEO WSR Coagulant | 10 | 10 | 10 |
| HPMC K4M | 10 | 10 | 10 |
| Lactose monohydrate | — | 35.7 | 25.65 |
| Microcrystalline Cellulose | 51.3 | 35.7 | 25.65 |
| Magnesium Stearate | 1 | 1 | 1 |
| Pluronic ® F127 | 20 | — | 20 |
| Dissolution (% release) | | | |
| Time (hrs) | | | |
| 0.5 | 11 | 13 | 12 |
| 2 | 18 | 19 | 20 |
| 4 | 25 | 23 | 30 |
| 8 | 45 | 33 | 67 |
| 12 | 73 | 47 | 95 |
| 16 | 94 | 62 | 98 |

### Example 2

The following examples are similar to those presented in Example 1, but use a higher viscosity grade of HPMC (100,000 cps)

TABLE 2

Carvedilol
example
formulations

| Components of Tablet | EO979 | EO790 |
|---|---|---|
| Formulation (%) | (%) | (%) |
| Carvedilol | 7.7 | 7.7 |
| PEO WSR Coagulant | 10 | 10 |
| HPMC K100M | 10 | 10 |
| Lactose monohydrate | 25.65 | — |
| Microcrystalline Cellulose | 25.65 | 71.3 |
| Magnesium Stearate | 1 | 1 |
| Pluronic ® F127 | 20 | — |
| Dissolution (% release) | | |
| Time (hrs) | | |
| 0.5 | 8 | 14 |
| 2 | 14 | 20 |
| 4 | 21 | 24 |
| 8 | 40 | 30 |
| 12 | 71 | 36 |
| 16 | 95 | 45 |

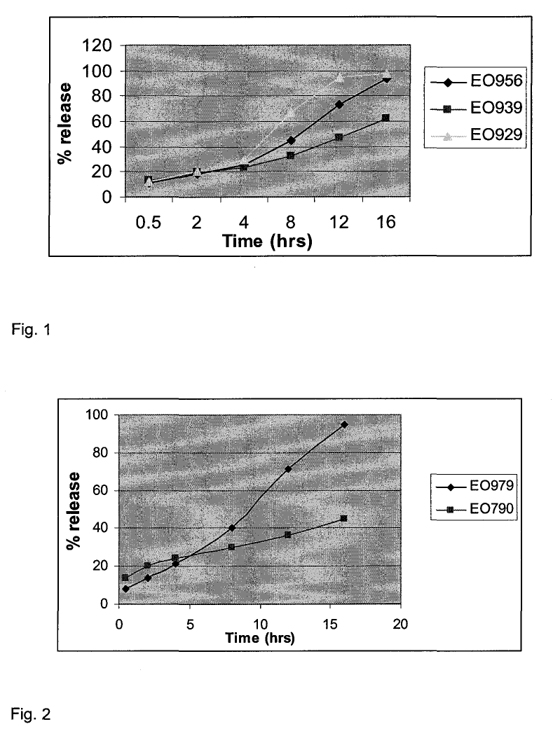

The benefit of incorporating Pluronic® into a PEO/HPMC based matrix tablet is clearly demonstrated from FIGS. 1 and 2, irrespective of whether a higher (K100M) or lower (K4M) grade of HPMC is employed. The data also demonstrates that the influence of Pluronic® cannot be substituted by a filler such as lactose or a microcrystalline cellulose.

As a result, by the incorporation of Pluronic® as a drug release modifier, drug release can be achieved whilst utilizing the benefits of a higher molecular weight PEO in combination with a high viscosity HPMC to maximize swelling, tablet integrity and therefore in-vivo gastric retention.

### Example 3

The following table provides examples of formulations of different drug potency comprising carvedilol and Pluronic®. The formulations shown below were prepared by first granulating the drug with a binder (in this case polyvinyl alcohol) to aid powder flow during compression.

TABLE 3

| Component | Composition (mg/Tablet and % w/w) | | | | | | | | | | | |
|---|---|---|---|---|---|---|---|---|---|---|---|---|
| Compendial | 6.25 mg | | 12.5 mg | | 25 mg | | 50 mg | | 75 mg | | 100 mg | |
| Name | mg | % | mg | % | mg | % | mg | % | mg | % | mg | % |
| Carvedilol | 6.25 | 0.96 | 12.50 | 1.92 | 25.00 | 3.85 | 50.00 | 7.69 | 75.00 | 11.54 | 100.01 | 14.29 |
| Polyethylene oxide | 65.00 | 10.00 | 65.00 | 10.00 | 65.00 | 10.00 | 65.00 | 10.00 | 65.00 | 10.00 | 70.00 | 10.00 |
| Hypromellose | 65.00 | 10.00 | 65.00 | 10.00 | 65.00 | 10.00 | 65.00 | 10.00 | 65.00 | 10.00 | 70.00 | 10.00 |
| Pluronic ® F127 | 130.00 | 20.00 | 130.00 | 20.00 | 130.00 | 20.00 | 130.00 | 20.00 | 130.00 | 20.00 | 140.00 | 20.00 |
| Microcrystalline | 188.24 | 28.96 | 184.79 | 28.43 | 177.19 | 27.26 | 165.88 | 25.52 | 153.01 | 23.54 | 154.84 | 22.12 |
| cellulose | | | | | | | | | | | | |
| Lactose Monohydrate | 188.30 | 28.97 | 184.79 | 28.43 | 178.46 | 27.46 | 165.94 | 25.53 | 152.97 | 23.53 | 154.79 | 22.11 |
| Polyvinyl Alcohol | 0.71 | 0.11 | 1.42 | 0.22 | 2.85 | 0.44 | 1.68 | 0.26 | 2.52 | 0.39 | 3.36 | 0.48 |
| Magnesium Stearate | 6.50 | 1.00 | 6.50 | 1.00 | 6.50 | 1.00 | 6.50 | 1.00 | 6.50 | 1.00 | 7.00 | 1.00 |
| Total | 650.00 | 100.00 | 650.00 | 100.00 | 650.00 | 100.01 | 650.00 | 100.00 | 650.00 | 100.00 | 700.00 | 100.00 |

### Dissolution Method:

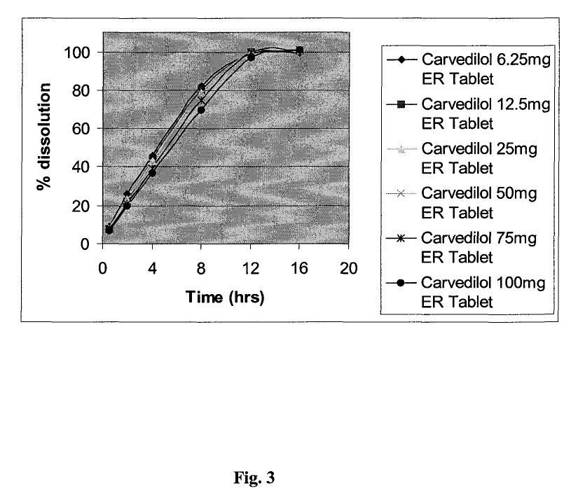

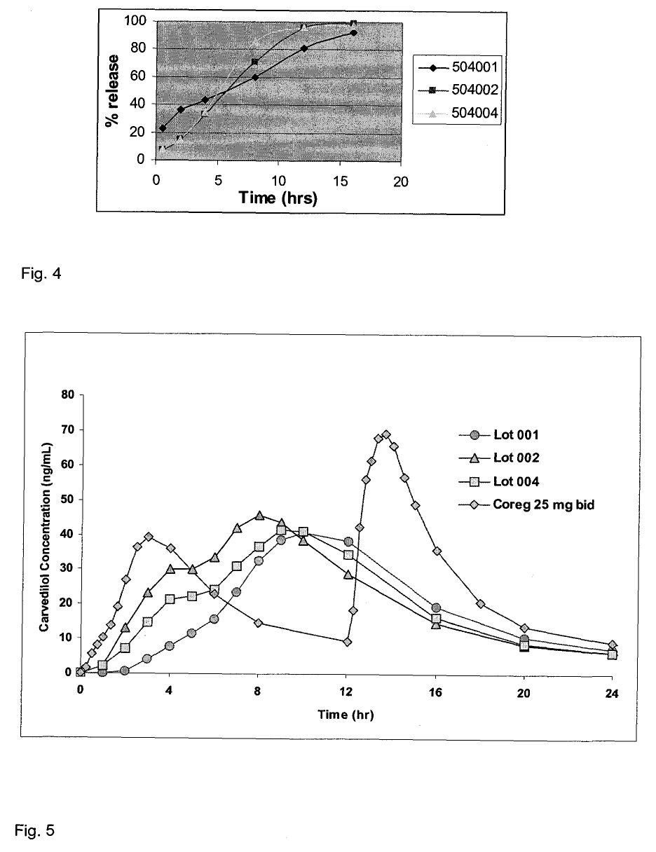

For the above formulations the data presented in FIG. 3 was generated using a USP II paddle apparatus with a stationary basket device as described in the USP Monograph for Felodopine Extended Release Tablets. In brief, in the dissolution method used, a quadrangular basket is suspended from a dissolution vessel lid and is positioned such that the bottom of the basket is 10 mm±1 mm from the top of the paddle; the paddle rotates as normal below the level of the basket. A hinged lid allows the tablet to be placed inside the basket and prevents the tablet from floating out. The dimensions of the basket restrict the tablet from rotating once placed inside. Dissolution was performed in 900 ml pH 4.5 acetate buffer with a paddle speed of 150 rpm.

Dissolution Data for Formulations in Table 3:

| | Carvedilol | Carvedilol | Carvedilol | Carvedilol | Carvedilol | Carvedilol |
|---|---|---|---|---|---|---|
| | 6.25 mg | 12.5 mg | 25 mg | 50 mg | 75 mg | 100 mg |
| Dissolution (hrs) | ER Tablet | ER Tablet | ER Tablet | ER Tablet | ER Tablet | ER Tablet |
| 0.5 | 9 | 7 | 7 | 9 | 8 | 7 |
| 2 | 26 | 22 | 23 | 23 | 21 | 20 |
| 4 | 46 | 44 | 43 | 42 | 39 | 37 |
| 8 | 82 | 80 | 79 | 78 | 75 | 70 |
| 12 | 99 | 100 | 100 | 100 | 100 | 97 |
| 16 | 100 | 100 | 100 | 100 | 100 | 100 |

### Example 4

The following table sets out some prophetic examples of gastric retentive formulations according to the present invention. The following formulations are of different drug potency and can be made by direct compression, i.e. in the absence of polyvinyl alcohol. The skilled person will appreciate that the formulations set out below will demonstrate that the rate and extent of drug dissolution is independent of drug potency in the formulation.

TABLE 4

| | Composition (mg/Tablet and % w/w) | | | | | | | | | | | |
|---|---|---|---|---|---|---|---|---|---|---|---|---|
| Compendial | 6.25 mg | | 12.5 mg | | 25 mg | | 50 mg | | 75 mg | | 100 mg | |
| Name | mg | % | mg | % | mg | % | mg | % | mg | % | mg | % |
| PEO Coagulant | 65.00 | 10.00 | 65.00 | 10.00 | 65.00 | 10.00 | 65.00 | 10.00 | 65.00 | 10.00 | 70.00 | 10.00 |
| HPMC K15M | 65.00 | 10.00 | 65.00 | 10.00 | 65.00 | 10.00 | 65.00 | 10.00 | 65.00 | 10.00 | 70.00 | 10.00 |
| Pluronic ® F127 | 130.00 | 20.00 | 130.00 | 20.00 | 130.00 | 20.00 | 130.00 | 20.00 | 130.00 | 20.00 | 140.00 | 20.00 |
| Microcrystalline | 188.95 | 29.07 | 186.21 | 28.65 | 180.04 | 27.7 | 167.56 | 25.78 | 155.53 | 23.93 | 158.2 | 22.6 |
| cellulose | | | | | | | | | | | | |
| Lactose Monohydrate | 188.30 | 28.97 | 184.79 | 28.43 | 178.46 | 27.46 | 165.94 | 25.53 | 152.97 | 23.53 | 154.79 | 22.11 |
| Magnesium Stearate | 6.50 | 1.00 | 6.50 | 1.00 | 6.50 | 1.00 | 6.50 | 1.00 | 6.50 | 1.00 | 7.00 | 1.00 |
| Total | 650.00 | 100.00 | 650.00 | 100.00 | 650.00 | 100.01 | 650.00 | 100.00 | 650.00 | 100.00 | 700.00 | 100.00 |

### Example 5

The following table sets out some prophetic examples of formulations according to the present invention. The formulations set out below illustrate various combinations of grades of PEO, HPMC and Poloxamer that can be used.

TABLE 5

| | 1 | 2 | 3 | 4 | 5 | 6 | 7 |
|---|---|---|---|---|---|---|---|
| Wt/mg based on 650 mg tablet | % w/w | % w/w | % w/w | % w/w | % w/w | % w/w | % w/w |
| Carvedilol | 7.7 | 7.7 | 7.7 | 7.7 | 7.7 | 7.7 | 7.7 |
| PEO WSR N-60K | — | 20 | — | — | — | 15 | — |
| PEO WSR Coagulant | 10 | — | 15 | 10 | 10 | 10 | 30 |
| Methocel K100M | — | — | 15 | 15 | 10 | — | — |
| Methocel K15M | — | — | — | — | — | — | 10 |
| Methocel K4M | 10 | 20 | — | — | — | 15 | — |
| Pluronic ® F68 | 20 | — | — | 7.7 | 20.5 | — | — |
| Pluronic ® F127 | — | 20 | 20 | — | — | 10 | 20 |
| Avicel ® pH 101 | 51.3 | 15.65 | 20.65 | 58.6 | 63.5 | 41.3 | 15.65 |
| Lactose Monohydrate | — | 15.65 | 20.65 | — | — | | 15.65 |
| Magnesium Stearate | 1.0 | 1.0 | 1.0 | 1.0 | 1.0 | 1.0 | 1.0 |

### Example 6

The following formulations were dosed in two PK biostudies as described below.

TABLE 6

| | Lot # of IMP Tablets | | | | | |
|---|---|---|---|---|---|---|
| | 0504001 | | 0504002 | | 0504004 | |
| Components of Tablet Formulation (%) | (%) | | (%) | | (%) | |
| 10025 API | 7.7 | | 7.7 | | 7.7 | |
| PEO WSR N-60K | 10 | | — | | — | |
| PEO WSR Coagulant | — | | 10 | | 10 | |
| Methocel K100M | — | | — | | 10 | |
| Methocel K4M | 10 | | 10 | | — | |
| Pluronic ® F127 | — | | 20 | | 20 | |
| Avicel ® pH 101 | 71.3 | | 25.65 | | 25.65 | |
| Lactose Monohydrate | — | | 25.65 | | 25.65 | |
| Magnesium Stearate | 1.0 | | 1.0 | | 1.0 | |
| Wt/mg based on 650 mg tablet | mg/tablet | % w/w | mg/tablet | % w/w | mg/tablet | % w/w |
| 10025 API | 50.05 | 7.7 | 50.05 | 7.7 | 50.05 | 7.7 |
| PEO WSR N-60K | 65.00 | 10 | — | — | — | — |
| PEO WSR Coagulant | — | — | 65.00 | 10 | 65.00 | 10 |
| Methocel K100M | — | — | — | — | 65.00 | 10 |
| Methocel K4M | 65.00 | 10 | 65.00 | 10 | — | — |
| Pluronic ® F127 | — | — | 130.00 | 20 | 130.00 | 20 |
| Avicel ® pH 101 | 463.45 | 71.3 | 166.73 | 25.65 | 166.73 | 25.65 |
| Lactose Monohydrate | — | — | 166.73 | 25.65 | 166.73 | 25.65 |
| Magnesium Stearate | 6.50 | 1.0 | 6.50 | 1.0 | 6.50 | 1.0 |

Dissolution data is presented below

TABLE 7

| Time (hrs) | 0504001 | 0504002 | 504004 |
|---|---|---|---|
| 0.5 | 23 | 7 | 8 |
| 2 | 36 | 15 | 15 |
| 4 | 43 | 33 | 35 |
| 8 | 60 | 71 | 86 |
| 12 | 81 | 96 | 96 |
| 16 | 93 | 99 | 98 |

### PK Data

### Study 1

### Objective

A pilot three-way, crossover, open-label, single-dose, fed, comparative bioavailability study of 3 carvedilol ER 50 mg tablets versus Coreg 25 mg tablets (50 mg dose b.i.d.) in normal healthy non-smoking male and female subjects.

The intent of this study was to evaluate the relative bioavailability of carvedilol from three test formulations under clinically relevant fed conditions.

### Study Design

The pilot study followed a single-dose, open-label, three-way, crossover design. The treatments were separated by a one (1) week washout period. All meals followed AHA (American Heart Association) specified nutrient content requirements.

Subjects received one of the following treatments during each study period, according to a computer generated randomization scheme:

### Treatment A (Lot 00504001)—See Example 6 Above for Formulation:

Following a fast of at least 4 hours, one Carvedilol 50 mg Tablet (starting at 20:00 hrs) with 240 mL of water 30 minutes after the start of an American Heart Association Meal.

(Treatment dose=50 mg).

### Treatment B (Lot 0504002):—See Example 6 Above for Formulation

Following a fast of at least 4 hours, one Carvedilol 50 mg Tablet (starting at 20:00 hrs) with 240 mL of water 30 minutes after the start of an American Heart Association Meal.

(Treatment dose=50 mg).

### Treatment C (Lot 0504004):—See Example 6 Above for Formulation

Following a fast of at least 4 hours, one Carvedilol 50 mg Tablet (starting at 20:00 hrs) with 240 mL of water 30 minutes after the start of an American Heart Association Meal.

(Treatment dose=50 mg).

### Treatment D (Coreg 25 mg):

Following a fast of at least 4 hours, one Coreg 25 mg Tablet (starting at 20:00 hrs) with 240 mL of water 30 minutes after the start of an American Heart Association Meal. Following an overnight of at least 7 hours, a second dose of one Coreg 25 mg Tablet (starting at 08:00 hrs, 12 hours post first dose), with 240 mL of ambient temperature water 30 minutes after the start of an American Heart Association breakfast. (Treatment dose=50 mg).

A total of 20 subjects were admitted into the study and all 20 subjects completed the study.

### Results

The data presented in Table 8, illustrates the comparative in-vitro dissolution of carvedilol from the three tested formulations. Formulation A possesses a significantly higher ‘burst release’ associated with the use of a lower molecular weight PEO in comparison with formulations B and C.

The ratio of geometric means (compared to Coreg) for carvedilol Cmax were 0.57, 0.74 and 0.66 for Formulations A, B and C respectively (Table 8). The ratio of geometric means (compared to Coreg) for carvedilol AUC₀₋ₜ were 0.75 and 0.86 and 0.81 for Formulations A, B and C respectively. Similarly the ratio of geometric means (compared to Coreg) for carvedilol AUC_{0-inf} were 0.76, 0.87 and 0.82 for Formulations A, B and C respectively.

The results demonstrate that, (i) surprisingly, with the inclusion of polyoxyalkylene copolymer as a drug release modifier, a greater extent of drug absorption is achievable from formulations comprising a higher molecular weight PEO than a formulation without polyoxyalkylene copolymer and a lower PEO molecular weight, (ii) even more surprisingly, a higher molecular weight PEO together with a higher viscosity HPMC delivers a greater extent of drug absorption than used in a formulation without a polyoxyalkylene copolymer and a lower PEO molecular weight.

TABLE 8
Summary of Mean Pharmacokinetic Parameters for Carvedilol

| | A | B | C | |
|---|---|---|---|---|
| PK | Carvedilol | Carvedilol | Carvedilol | |
| Parameters | 50 mg | 50 mg | 50 mg | Coreg 25 mg |
| (n = 20) | (Lot 0504001) | (Lot 0504002) | (Lot 0504004) | b.i.d |
| AUC0-t | 509.87 +/− 237.49 | 583.96 +/− 271.56 | 550.7 +/− 247.93 | 675.05 +/− 328.84 |
| (ng * hr/mL) | | | | |
| AUC0-inf | 531.9 +/− 261.22 | 588.67 +/ 287.68 | 571.56 +/ 264.11 | 690.25 +/− 344.68 |
| (ng * hr/mL) | | | | |
| Cmax | 48.11 +/− 20.72 | 61.52 +/− 24.85 | 58.82 +/− 32.85 | 81.51 +/− 32.64 |
| (ng/mL) | | | | |
| Tmax (hr)* | 9.65 +/− 1.66 | 6.85 +/− 2.7 | 8.25 +/− 2.36 | 1.45 +/− 0.79 |
| t½ | 9.17 +/− 5.69 | 10.02 +/− 5.52 | 10.39 +/− 6.52 | 7.75 +/− 3.81 |
| MRT | 14.61 +/− 2.12 | 12.29 +/− 2.22 | 13.11 +/− 2.27 | 13.53 +/− 1.84 |

*Median Tmax,
**p < 0.05

TABLE 9

| | PK Parameter | | | | | |
|---|---|---|---|---|---|---|
| | AUC0-t | | AUC0-inf | | Cmax | |
| Comparison | Ratio | 90% CI | Ratio | 90% CI | Ratio | 90% CI |

Moiety: Carvedilol

| | | | | | | |
|---|---|---|---|---|---|---|
| A vs Coreg | 0.75 | 69-81 | 0.76 | 70-83 | 0.57 | 51-64 |
| B vs Coreg | 0.86 | 79-94 | 0.87 | 81-95 | 0.74 | 66-83 |
| C vs Coreg | 0.81 | 75-88 | 0.82 | 75-89 | 0.66 | 59-74 |

### Example 7

The following two formulations demonstrate an alternative embodiment of the dosage form according to the invention.

EO939 is a 650 mg 17 mm×9 mm tablet matrix formulation comprising containing 50 mg carvedilol and 10% w/w 5,000,000 MW Polyethylene oxide (PEO WSR Coag.) and 10% w/w 4,000 cps HPMC (K4M).

EO938 is a tablet identical in size and shape to EO939, has the same levels of K4M and PEO WSR Coag., but differs in that the drug is present in a 30:70 drug:Eudragit® E solid dispersion.

### Dissolution Method:

Dissolution was performed in a US Pharmacopeia 27 dissolution apparatus II (paddles). Given the swellable and potentially floatable nature of the carvedilol tablets, tablets were loosely wrapped in copper wire (≦165 mm×0.5 mm) to hold them in place without restricting swelling. Dissolution was performed at 75 rpm in 900 ml of 0.1N HCl+5% Tween.

TABLE 10

Carvedilol
example
formulations

| Components of Tablet | EO938 | EO939 |
|---|---|---|
| Formulation (%) | (%) | (%) |
| Carvedilol | — | 7.7 |
| Carvedilol/Eudragit ® 30:70 | 25.6 | |
| extrudate | | |
| PEO WSR Coagulant | 10 | 10 |
| HPMC K4M | 10 | 10 |
| Lactose monohydrate | 26.7 | 35.7 |
| Microcrystalline Cellulose | 26.7 | 35.7 |
| Magnesium Stearate | 1 | 1 |
| Dissolution (% release) | | |
| Time (hrs) | | |
| 0.5 | 5 | 13 |
| 2 | 13 | 19 |
| 4 | 24 | 23 |
| 8 | 60 | 33 |
| 12 | 89 | 47 |
| 16 | 98 | 62 |

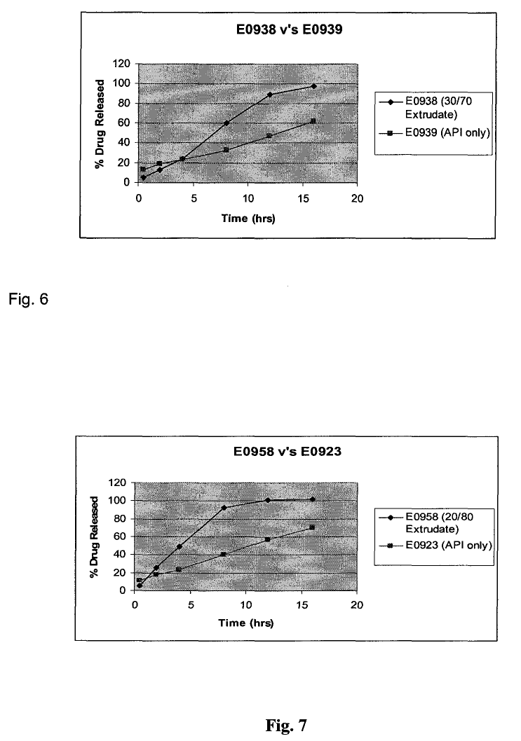

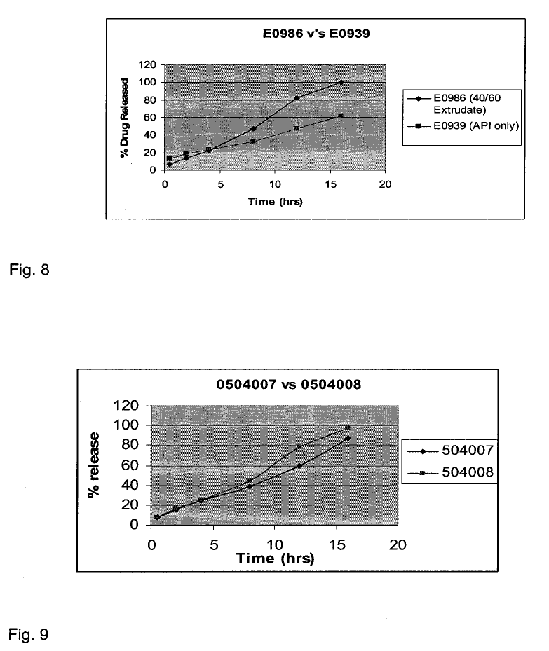

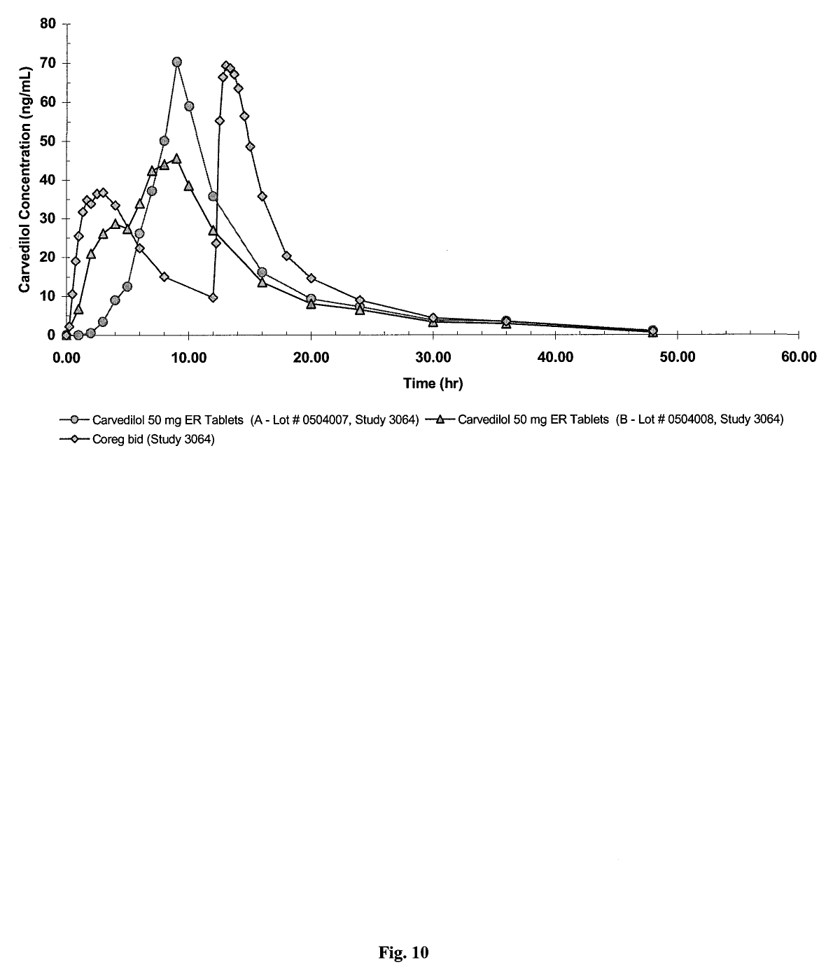

As shown in FIG. 6, the formulation comprising Carvedilol/Eudragit® 30:70 solid dispersion demonstrates a 98% release of carvedilol after 16 hours compared to a 62% release for when carvedilol alone is used.

### Example 8

The following examples are similar to Example 7, but use a carvedilol:Eudragit® E ratio of 20:80 in the formation of the solid dispersion.

TABLE 11

Carvedilol
example
formulations

| | EO958 | EO923 |
|---|---|---|
| | (%) | (%) |
| Components of Tablet | | |
| Formulation (%) | | |
| Carvedilol | — | 77 |
| Carvedilol/Eudragit ® 20:80 | 38.5 | |
| extrudate | | |
| PEO WSR Coagulant | 10 | 10 |
| HPMC K4M | 10 | 10 |
| Pluronic ® F127 | 10 | 10 |
| Microcrystalline Cellulose | 30.5 | 31.3 |
| Magnesium Stearate | 1 | 1 |
| Dissolution (% release) | | |
| Time (hrs) | | |
| 0.5 | 5 | 11 |
| 2 | 26 | 18 |
| 4 | 49 | 23 |
| 8 | 93 | 40 |
| 12 | 100 | 57 |
| 16 | | 70 |

### Example 9

The following examples are similar to Example 7, but use a carvedilol:Eudragit® E ratio of 40:60 in the formation of the solid dispersion.

TABLE 12

Carvedilol
example
formulations

| | EO986 | EO939 |
|---|---|---|
| | (%) | (%) |
| Components of Tablet | | |
| Formulation (%) | | |
| Carvedilol | — | 7.7 |
| Carvedilol/Eudragit ® 40:60 | 19.25 | |
| extrudate | | |
| PEO WSR Coagulant | 10 | 10 |
| HPMC K4M | 10 | 10 |
| Lactose monohydrate | 29.9 | 35.7 |
| Microcrystalline Cellulose | 29.9 | 35.7 |
| Magnesium Stearate | 1 | 1 |
| Dissolution (% release) | | |
| Time (hrs) | | |
| 0.5 | 7 | 13 |
| 2 | 14 | 19 |
| 4 | 22 | 23 |
| 8 | 47 | 33 |
| 12 | 82 | 47 |
| 16 | 100 | 62 |

### Example 10

The following table sets out some prophetic examples of gastrically retained formulations according to the present invention comprising a solid dispersion of carvedilol and Eudragit® E.

TABLE 13

| Wt/mg based on | 1 | 2 | 3 | 4 | 5 |
|---|---|---|---|---|---|
| 650 mg tablet | % w/w | % w/w | % w/w | % w/w | % w/w |
| Carvedilol | — | — | — | 19.25 | 19.25 |
| (40% w/w)/Eudragit ® | | | | | |
| (60% w/w) extrudate | | | | | |
| Carvedilol | 25.6 | — | 25.6 | — | — |
| (30% w/w)/Eudragit ® | | | | | |
| (70% w/w) extrudate | | | | | |
| Carvedilol | — | 38.5 | — | — | — |
| (20% w/w)/Eudragit ® | | | | | |
| (80% w/w) extrudate | | | | | |
| PEO WSR N-60K | 10 | — | 20 | 25 | 30 |
| PEO WSR Coagulant | — | 10 | — | — | — |
| Methocel K100M | 10 | — | — | 25 | — |
| Methocel K15M | — | — | 10 | — | — |
| Methocel K4M | — | 20 | — | — | 20 |
| Pluronic ® F68 | — | — | — | — | — |
| Pluronic F127 | — | 10 | — | — | — |
| Avicel ® pH 101 | 53.4 | 15.25 | 43.4 | 29.75 | 19.75 |
| Lactose Monohydrate | — | 5.25 | — | — | 10 |
| Magnesium Stearate | 1.0 | 1.0 | 1.0 | 1.0 | 1.0 |

### Example 11

The following formulations were dosed in a pharmacokinetic (PK) study.

TABLE 14

| | Lot # of IMP Tablets | | | |
|---|---|---|---|---|
| Components of Tablet | 0504008 | | 0504007 | |
| Formulation (%) | (%) | | (%) | |
| carvedilol/EPO 40/60 | 19.25 | | — | |
| Extrudate | | | | |
| carvedilol/EPO 30/70 | — | | 25.6 | |
| Extrudate | | | | |
| PEO WSR Coagulant | 10.0 | | 10.0 | |
| Methocel K100M | — | | 10.0 | |
| Methocel K4M | 10.0 | | — | |
| Avicel ® pH 101 | 29.9 | | 53.4 | |
| Lactose Monohydrate | 29.9 | | — | |
| Magnesium Stearate | 1.0 | | 1.0 | |
| Wt/mg | | | | |
| based on 650 mg tablet | mg/tablet | % w/w | mg/tablet | % w/w |
| carvedilol API | 50.05 | 7.7 | 50.05 | 7.7 |
| EPO | 74.75 | 11.5 | 116.35 | 17.9 |
| PEO WSR Coagulant | 65.00 | 10.0 | 65.00 | 10.0 |
| Methocel K100M | — | — | 65.00 | 10.0 |
| Methocel K4M | 65.00 | 10.0 | — | — |
| Avicel ® pH 101 | 194.35 | 29.9 | 347.10 | 53.4 |
| Lactose Monohydrate | 194.35 | 29.9 | — | — |
| Magnesium Stearate | 6.50 | 1.0 | 6.50 | 1.0 |

Dissolution data for each of the above formulations is presented below:

TABLE 15

| Time (hrs) | 0504008 | 0504007 |
|---|---|---|
| 0.5 | 8 | 8 |
| 2 | 17 | 15 |
| 4 | 25 | 24 |
| 8 | 44 | 39 |
| 12 | 78 | 59 |
| 16 | 97 | 87 |

### PK Data

A pilot three-way, crossover, open-label, single-dose, fed, comparative bioavailability study of carvedilol ER 50 mg 40 mg tablets versus Coreg 25 mg tablets (25 mg B.I.D.) in normal healthy non-smoking male and female subjects.

The intent of this study was to evaluate the relative bioavailability of carvedilol from two test formulations under clinically relevant fed conditions.

### Study Design

This pilot study followed a single-dose, open-label, three-way, crossover design. The treatments were separated by a one (1) week washout period. All meals followed AHA (American Heart Association) specified nutrient content requirements.

Subjects received one of the following treatments during each study period, according to a computer generated randomization scheme:

### Treatment A (Lot 00504007)—See Example 11 for Formulation:

Following a fast of at least 4 hours, one Carvedilol 50 mg Tablet (starting at 20:00 hrs) with 240 mL of water 30 minutes after the start of an American Heart Association Meal.

(Treatment dose=50 mg).

### Treatment B (Lot 0504008):—See Example 11 for Formulation

Following a fast of at least 4 hours, one Carvedilol 50 mg Tablet (starting at 20:00 hrs) with 240 mL of water 30 minutes after the start of an American Heart Association Meal.

(Treatment dose=40 mg).

### Treatment C (Coreg 25 mg):

Following a fast of at least 4 hours, one Coreg 25 mg Tablet (starting at 20:00 hrs) with 240 mL of water 30 minutes after the start of an American Heart Association Meal. Following an overnight of at least 7 hours, a second dose of one Coreg 25 mg Tablet (starting at 08:00 hrs, 12 hours post first dose), with 240 mL of ambient temperature water 30 minutes after the start of an American Heart Association breakfast. (Treatment dose=50 mg).

A total of 21 subjects were admitted into the study and 20 subjects completed the study.

The ratio of geometric means (compared to Coreg) for carvedilol Cmax were 0.95 and 0.87 for Formulations A and B respectively. The ratio of geometric means (compared to Coreg) for carvedilol AUC₀₋ₜ were 0.85 and 0.84 for Formulations A and B respectively. Similarly the ratio of geometric means (compared to Coreg) for carvedilol AUC_{0-inf} were 0.86 and 0.83 for Formulations A and B respectively.

TABLE 16
Summary of Mean Pharmacokinetic Parameters for Carvedilol

| | A | | |
|---|---|---|---|
| PK | Carvedilol | B | |
| Parameters | 50 mg | Carvedilol 50 mg | |
| (n = 20) | (Lot 0504007) | (Lot 0504008) | Coreg 25 mg b.i.d |
| AUC0-t | 595.78 +/− | 584.33 +/− 313.6 | 696.91 +/− 360.7 |
| (ng * hr/mL) | 317.21 | | |
| AUC0-inf | 613.75 +/− | 593.54 +/− 330.46 | 711.72 +/− 387.92 |
| (ng * hr/mL) | 337.81 | | |
| Cmax | 80.01 +/− | 70.84 +/− 27.27 | 83.62 +/− 34.15 |
| (ng/mL) | 38.01 | | |
| Tmax (hr)* | 8.45 1.28 | 6.5 2.31 | 11.41 +/− 4.47 |
| t½ | 8.63 +/− 3.98 | 7.57 +/− 2.52 | 7.17 +/− 3.27 |
| MRT | 13.76 +/− | 11.68 +/− 2.49 | 13.33 +/− 1.8 |
| | 1.42 | | |

*Median Tmax
**p < 0.05

TABLE 17

| | PK Parameter | | | | | |
|---|---|---|---|---|---|---|
| | AUC0-t | | AUC0-inf | | Cmax | |
| Comparison | Ratio | 90% CI | Ratio | 90% CI | Ratio | 90% CI |

Moiety: Carvedilol

| | | | | | | |
|---|---|---|---|---|---|---|
| A vs Coreg | 0.85 | 79-93 | 0.86 | 80-94 | 0.95 | 84-107 |
| B vs Coreg | 0.84 | 77-90 | 0.83 | 77-90 | 0.87 | 77-99 |

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

1) A controlled release dosage form comprising a therapeutically effective amount of carvedilol and/or a pharmaceutically acceptable salt thereof; one or more hydrophilic polymers; one or more pharmaceutical excipients and a polyoxyalkylene block copolymer.

2) The controlled release dosage form of claim 1 wherein said polyoxyalkylene block copolymer comprises a block copolymer of ethylene oxide:propylene oxide:ethylene oxide.

3) The controlled release dosage form of claim 1 wherein said polyoxyalkylene block copolymer comprises an α-hydro-ω-hydroxypropyl(oxyethylene)poly(oxypropylene)poly(oxyethylene) block copolymer.

4) The controlled release dosage form of claim 1 wherein said polyoxyalkylene block copolymer is selected from the group consisting of HO(C₂H₄O)₈₀(C₃H₆O)₂₇(C₂H₄O)₈₀H, HO(C₂H₄O)₁₀₁ (C₃H₆O)₅₆(C₂H₄O)₁₀₁H, HO(C₂H₄O)₆₄(C₃H₆O)₃₇(C₂H₄O)₆₄H, HO(C₂H₄O)₁₄₁(C₃H₆O)₄₄(C₂H₄O)₁₄₁H and any combination thereof.

5) The controlled release dosage form of claim 1 wherein said one or more hydrophilic polymers are selected from the group consisting of polyethylene oxide, hydroxypropyl methylcellulose, hydroxyethyl cellulose, hydroxypropyl methylcellulose, hydroxypropyl methylcellulose phthalate, methylcellulose and any combination thereof.

6) The controlled release dosage form of claim 1 wherein said hydrophilic polymers comprise polyethylene oxide and hydroxypropyl methylcellulose.

7) The controlled release dosage form of claim 6 wherein said polyethylene oxide has a molecular weight in the range of 900,000 to 10,000,000.

8) The controlled release dosage form of claim 6 wherein said hydroxypropyl methylcellulose has a viscosity in the range from about 4,000 centipoise to about 1,000,000 centipoise when measured as a 2% solution in water at 20° C.

9) The controlled release dosage form of claim 1 wherein said dosage form comprise a crystalline form of said carvedilol.

10) The controlled release dosage form of claim 1 wherein said dosage form comprises an amorphous form of said carvedilol.

11) The controlled release dosage form of claim 1 wherein said dosage form comprises a combination of an amorphous and crystalline form of carvedilol.

12) The controlled release dosage form of claim 1 further comprising a diluent.

13) The controlled release dosage form of claim 12 wherein the diluent is selected from the group consisting of lactose, microcrystalline cellulose, carboxymethylcellulose calcium, calcium phosphate, calcium sulfate, hydrogenated vegetable oils, sugar, glyceryl palmitostearate, pregelatinised starch, sorbitol, maltitol and any combination thereof.

14) The controlled release dosage form of claim 1 further comprising a lubricant.

15) The controlled release dosage form of claim 14 wherein said lubricant is selected from the group consisting of magnesium stearate, sodium stearyl fumarate, colloidal silicon dioxide, glyceryl monostearate, glyceryl dibehanate, talc and any combination thereof.

16) The controlled release dosage form of claim 1 wherein said dosage form comprises a tablet.

17) The controlled release dosage form of claim 1 wherein said dosage form comprises a capsule.

18) The controlled release dosage form of claim 1 wherein said dosage form comprises:
a) carvedilol in an amount from about 0.1 to about 50% by weight;
b) polyethylene oxide in an amount from about 1 to about 60% by weight;
c) hydroxypropyl methylcellulose in an amount from about 1 to about 60% by weight;
d) lactose in an amount from about 0 to about 80% by weight;
e) microcrystalline cellulose in an amount from about 1 to about 99% by weight;
f) magnesium stearate in an amount from about 0.05 to about 10% by weight; and
g) polyalkylene block copolymer in an amount from about 5 to about 40% by weight.

19) The controlled release dosage form of claim 1 wherein said dosage form comprises:
a) carvedilol in an amount of about 7.7% by weight;
b) polyethylene oxide in an amount of about 10% by weight;
c) hydroxypropyl methylcellulose in an amount of about 10% by weight;
d) lactose in an amount of about 25.65% by weight;
e) microcrystalline cellulose in an amount of about 25.65% by weight;
f) magnesium stearate in an amount of about 1% by weight; and
g) polyalkylene block copolymer in an amount of about 20% by weight.

20) The controlled release dosage form of claim 1 wherein said dosage form comprises a tablet comprising:

| | | % W/W |
|---|---|---|
| a) | carvedilol | 0.96 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 28.96 |
| f) | lactose monohydrate | 28.97 |
| g) | polyvinyl alcohol | 0.11 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 9% is released after about 0.5 hours; about 26% is released after about 2 hours; about 46% is released after about 4 hours; about 82% is released after about 8 hours; about 99% is released after about 12 hours and about 100% is released after about 16 hours.

21) The controlled release dosage form of claim 1 wherein said dosage form comprises a tablet comprising:

| | | % W/W |
|---|---|---|
| a) | carvedilol | 1.92 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 28.43 |
| f) | lactose monohydrate | 28.43 |
| g) | polyvinyl alcohol | 0.22 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 7% is released after about 0.5 hours; about 22% is released after about 2 hours; about 44% is released after about 4 hours; about 80% is released after about 8 hours; and about 100% is released after about 12 hours.

22) The controlled release dosage form of claim 1 wherein said dosage form comprises a tablet comprising:

| | | % W/W |
|---|---|---|
| a) | carvedilol | 3.85 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 27.26 |
| f) | lactose monohydrate | 27.46 |
| g) | polyvinyl alcohol | 0.44 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 7% is released after about 0.5 hours; about 23% is released after about 2 hours; about 43% is released after about 4 hours; about 79% is released after about 8 hours; and about 100% is released after about 12 hours.

23) The controlled release dosage form of claim 1 wherein said dosage form comprises a tablet comprising:

| | | % W/W |
|---|---|---|
| a) | carvedilol | 7.69 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 25.52 |
| f) | lactose monohydrate | 25.53 |
| g) | polyvinyl alcohol | 0.26 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 9% is released after about 0.5 hours; about 23% is released after about 2 hours; about 42% is released after about 4 hours; about 78% is released after about 8 hours; and about 100% is released after about 12 hours.

24) The controlled release dosage form of claim 1 wherein said dosage form comprises a tablet comprising:

| | | % W/W |
|---|---|---|
| a) | carvedilol | 11.54 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 23.54 |
| f) | lactose monohydrate | 23.53 |
| g) | polyvinyl alcohol | 0.39 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 8% is released after about 0.5 hours; about 21% is released after about 2 hours; about 39% is released after about 4 hours; about 75% is released after about 8 hours; and about 100% is released after about 12 hours.

25) The controlled release dosage form of claim 1 wherein said dosage form comprises a tablet comprising:

| | | % W/W |
|---|---|---|
| a) | carvedilol | 14.29 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | microcrystalline cellulose | 22.12 |
| f) | lactose monohydrate | 22.11 |
| g) | polyvinyl alcohol | 0.48 |
| h) | magnesium stearate | 1.00 |

wherein the dosage form is adapted to release the carvedilol into an acidic medium at the such that when measured using the method of United States Pharmacopoeia No. II at 150 rpm in 900 ml pH 4.5 acetate buffer about 7% is released after about 0.5 hours; about 20% is released after about 2 hours; about 37% is released after about 4 hours; about 70% is released after about 8 hours; about 97% is released after about 12 hours, and about 100% is released after about 16 hours.

26) The controlled release dosage form of claim 1 wherein said dosage form comprises a tablet comprising:

| | | % W/W |
|---|---|---|
| a) | carvedilol | 7.7 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | Microcrystalline cellulose | 25.65 |
| f) | lactose monohydrate | 25.65 |
| h) | magnesium stearate | 1.00 |

wherein said dosage tablet when administered to a subject in need of such administration, following a fast of at least about 4 hours, with 240 ml of water about 30 minutes after the start of an American Heart Association meal, exhibits a mean AUC_{(0-inf)} for carvedilol of about 588.67 ng*hr/ml after administration of a once daily 50 mg dose of said controlled release carvedilol dosage tablet.

27) The controlled release dosage form of claim 26 wherein said dosage form exhibits a mean AUC₍₀₋ₜ₎ for carvedilol of about 583.96 ng*hr/ml after administration of a once-daily 50 mg dose of said controlled release carvedilol tablet.

28) The controlled release dosage form of claim 26 wherein said dosage form exhibits a mean Cₘₐₓ for carvedilol of about 61.52 ng/ml after administration of a once-daily 50 mg dose of said controlled release carvedilol tablet.

29) The controlled release dosage form of claim 27 wherein said dosage form exhibits a mean Cₘₐₓ for carvedilol of about 61.52 ng/ml after administration of a once-daily 50 mg dose of said controlled release carvedilol tablet.

30) The controlled release dosage form of claim 28 wherein said dosage form exhibits a median Tₘₐₓ for carvedilol of about 6.85 hr after administration of a once daily 50 mg dose of said controlled release carvedilol tablet.

31) The controlled release dosage form of claim 29 wherein said dosage form exhibits a median Tₘₐₓ for carvedilol of about 6.85 hr after administration of a once daily 50 mg dose of said controlled release carvedilol tablet.

32) The controlled release dosage form of claim 1 wherein said dosage form comprises a tablet comprising:

| | | % W/W |
|---|---|---|
| a) | carvedilol | 7.7 |
| b) | polyethylene oxide | 10.00 |
| c) | hydroxypropyl methylcellulose | 10.00 |
| d) | HO(C₂H₄O)₁₄₁(C₃H₆O)₄₄(C₂H₄O)₁₄₁H | 20.00 |
| e) | Microcrystalline cellulose | 25.65 |
| f) | lactose monohydrate | 25.65 |
| h) | magnesium stearate | 1.00 |

wherein said dosage tablet when administered to a subject in need of such administration, following a fast of at least about 4 hours, with 240 ml of water about 30 minutes after the start of an American Heart Association meal, exhibits a mean AUC_{(0-inf)} for carvedilol of about 571.56 ng*hr/ml after administration of a once daily 50 mg dose of said controlled release carvedilol dosage tablet.

33) The controlled release dosage form of claim 32 wherein said dosage form exhibits a mean AUC₍₀₋ₜ₎ for carvedilol of about 550.7 ng*hr/ml after administration of a once-daily 50 mg dose of said controlled release carvedilol tablet.

34) The controlled release dosage form of claim 32 wherein said dosage form exhibits a mean Cₘₐₓ for carvedilol of about 58.82 ng/ml after administration of a once-daily 50 mg dose of said controlled release carvedilol tablet.

35) The controlled release dosage form of claim 33 wherein said dosage form exhibits a mean Cₘₐₓ for carvedilol of about 58.82 ng/ml after administration of a once-daily 50 mg dose of said controlled release carvedilol tablet.

36) The controlled release dosage form of claim 34 wherein said dosage form exhibits a median Tₘₐₓ for carvedilol of about 8.25 hr after administration of a once daily 50 mg dose of said controlled release carvedilol tablet.

37) The controlled release dosage form of claim 35 wherein said dosage form exhibits a median Tₘₐₓ for carvedilol of about 8.25 hr after administration of a once daily 50 mg dose of said controlled release carvedilol tablet.

38) The controlled release dosage form of claim 1 further comprising a coating.

39) The controlled release dosage form of claim 38 wherein said coating comprises a second drug.

40) The controlled release dosage form of claim 39 wherein said second drug is selected from the group consisting of ACE inhibitors, diuretics, digoxin and any combination thereof.

41) A method of treating a cardiovascular condition in a subject in need of such treatment comprising administering to said subject the controlled release dosage form of claim 1.

42) The method of claim 41 wherein said cardiovascular condition is selected from the group consisting of hypertension, congestive heart failure, left ventricular dysfunction following myocardial infarction and any combination thereof.

43) The method of claim 41 wherein said dosage form is administered once daily.

44) A controlled release dosage form comprising a therapeutically effective amount of carvedilol and/or a pharmaceutically acceptable salt thereof; one or more hydrophilic polymers; one or more pharmaceutically acceptable excipients, and a solid dispersion of carvedilol and an extrusion material.

45) The controlled release dosage form of claim 44 wherein said extrusion material comprises an extrusion polymer.

46) The controlled release dosage form of claim 44 wherein said extrusion material comprises a non-polymeric material.

47) The controlled release dosage form of claim 44 wherein said one or more hydrophilic polymers are selected from the group consisting of polyethylene oxide, hydroxypropyl methylcellulose, hydroxyethyl cellulose, hydroxypropyl methylcellulose, hydroxypropyl methylcellulose phthalate, methylcellulose and any combination thereof.

48) The controlled release dosage form of claim 44 wherein said hydrophilic polymers comprise polyethylene oxide and hydroxypropyl methylcellulose.

49) The controlled release dosage form of claim 48 wherein said polyethylene oxide has a molecular weight in the range of 900,000 to 10,000,000.

50) The controlled release dosage form of claim 48 wherein said hydroxypropyl methylcellulose has a viscosity in the range from about 4,000 centipoise to about 1,000,000 centipoise when measured as a 2% solution in water at 20° C.

51) The controlled release dosage form of claim 44, wherein said extrusion polymer is selected from the group consisting of a methacrylic acid ester terpolymeric product of butyl methacrylate, (2-dimethyl aminoethyl)methacrylate, and methyl methacrylate 1:2:1, polyethylene glycols, polyoxyethylene glycols, polyethylene-propylene glycol copolymers, polyethylene oxides, polyvinyl pyrrolidinone (also referred to as polyvinyl pyrrolidone or povidone or PVP), polyvinyl alcohol, polyethylenevinyl alcohol copolymers, polyvinyl alcohol polyvinyl acetate copolymers, xanthan gum, carrageenan, hydroxypropyl cellulose, hydroxypropyl methylcellulose, carboxymethyl cellulose, carboxymethyl ethyl cellulose, carboxylic acid-functionalized polymethacrylates, amine-functionalized polymethacrylates, chitosan, chitin, polydextrose, dextrin and starch, and any combination thereof.

52) The controlled release dosage form according to claim 44 wherein said extrusion polymer comprises a methacrylic acid ester terpolymeric product of butyl methacrylate, (2-dimethyl aminoethyl)methacrylate, and methyl methacrylate 1:2:1.

53) The controlled release dosage form according to claim 44 wherein the ratio of carvedilol: methyl methacrylate 1:2:1 is 20:80.

54) The controlled release dosage form according to claim 44 wherein the ratio of carvedilol: methyl methacrylate 1:2:1 is 30:70.

55) The controlled release dosage form according to claim 44 wherein the ratio of carvedilol: methyl methacrylate 1:2:1 is 40:60.

56) The controlled release dosage form of claim 44 wherein said dosage form comprises a crystalline form of said carvedilol.

57) The controlled release dosage form of claim 44 wherein said dosage form comprises an amorphous form of said carvedilol.

58) The controlled release dosage form of claim 44 wherein said dosage form comprises a combination of an amorphous and crystalline form of said carvedilol.

59) The controlled release dosage form of claim 44 further comprising a diluent.

60) The controlled release dosage form of claim 59 wherein the diluent is selected from the group consisting of lactose, microcrystalline cellulose, carboxymethylcellulose calcium, calcium phosphate, calcium sulfate, hydrogenated vegetable oils, sugar, glyceryl palmitostearate, pregelatinised starch, sorbitol, maltitol and any combination thereof.

61) The controlled release dosage form of claim 44 further comprising a lubricant.

62) The controlled release dosage form of claim 61 wherein said lubricant is selected from the group consisting of magnesium stearate, sodium stearyl fumarate, colloidal silicon dioxide, glyceryl monostearate, glyceryl dibehanate, talc and any combination thereof.

63) The controlled release dosage form of claim 44 wherein said dosage form comprises a tablet.

64) The controlled release dosage form of claim 44 wherein said dosage form comprises a capsule.

65) The controlled release dosage form of claim 44 wherein said dosage form comprises:
a) carvedilol in an amount from about 0.1 to about 50% by weight;
b) methyl methacrylate 1:2:1 in an amount from about 1% to about 50% by weight;
c) polyethylene oxide in an amount from about 1 to about 60% by weight;
d) hydroxypropyl methylcellulose in an amount from about 1 to about 60% by weight;
e) lactose in an amount from about 0 to about 80% by weight;
f) microcrystalline cellulose in an amount from about 1 to about 99% by weight; and
g) magnesium stearate in an amount from about 0.05 to about 10% by weight.

66) The controlled release dosage form of claim 44 wherein said dosage form comprises:
a) carvedilol in an amount of about 7.7% by weight;
b) methyl methacrylate 1:2:1 in an amount of about 11.5% by weight;
c) polyethylene oxide in an amount of about 10% by weight;
d) hydroxypropyl methylcellulose in an amount of about 10% by weight;
e) lactose in an amount of about 29.9% by weight;
f) microcrystalline cellulose in an amount of about 29.9% by weight; and
g) magnesium stearate in an amount of about 1% by weight.

67) The controlled release dosage form of claim 44 wherein said dosage form comprises a tablet comprising:

| | | % W/W |
|---|---|---|
| a) | Carvedilol/methyl methacrylate 1:2:1 | 19.25 |
| | 40:60 extrudate | |
| b) | polyethylene oxide | 10.00 |
| c) | hydroxypropyl methylcellulose | 10.00 |
| e) | microcrystalline cellulose | 29.9 |
| f) | lactose monohydrate | 29.9 |
| h) | magnesium stearate | 1.00 |

wherein said dosage tablet when administered to a subject in need of such administration, following a fast of at least about 4 hours, with 240 ml of water about 30 minutes after the start of an American Heart Association meal, exhibits a mean AUC_{(0-inf)} for carvedilol of about 593.54 ng*hr/ml after administration of a once daily 50 mg dose of said controlled release carvedilol dosage tablet.

68) The controlled release dosage form of claim 67 wherein said dosage form exhibits a mean AUC₍₀₋ₜ₎ for carvedilol of about 584.33 ng*hr/ml after administration of a once-daily 50 mg dose of said controlled release carvedilol tablet.

69) The controlled release dosage form of claim 67 wherein said dosage form exhibits a mean Cₘₐₓ for carvedilol of about 70.84 ng/ml after administration of a once-daily 50 mg dose of said controlled release carvedilol tablet.

70) The controlled release dosage form of claim 68 wherein said dosage form exhibits a mean Cₘₐₓ for carvedilol of about 70.84 ng/ml after administration of a once-daily 50 mg dose of said controlled release carvedilol tablet.

71) The controlled release dosage form of claim 69 wherein said dosage form exhibits a median Tₘₐₓ for carvedilol of about 6.5 hr after administration of a once daily 50 mg dose of said controlled release carvedilol tablet.

72) The controlled release dosage form of claim 70 wherein said dosage form exhibits a median Tₘₐₓ for carvedilol of about 6.5 hr after administration of a once daily 50 mg dose of said controlled release carvedilol tablet.

73) The controlled release dosage form of claim 44 wherein said dosage form comprises a tablet comprising:

| | | % W/W |
|---|---|---|
| a) | Carvedilol/methyl methacrylate 1:2:1 | 25.6 |
| | 30:70 extrudate | |
| b) | polyethylene oxide | 10.00 |
| c) | hydroxypropyl methylcellulose | 10.00 |
| e) | microcrystalline cellulose | 53.4 |
| h) | magnesium stearate | 1.00 |

wherein said dosage tablet when administered to a subject in need of such administration, following a fast of at least about 4 hours, with 240 ml of water about 30 minutes after the start of an American Heart Association meal, exhibits a mean AUC_{(0-inf)} for carvedilol of about 613.75 ng*hr/ml after administration of a once daily 50 mg dose of said controlled release carvedilol dosage tablet.

74) The controlled release dosage form of claim 73 wherein said dosage form exhibits a mean AUC₍₀₋ₜ₎ for carvedilol of about 595.78 ng*hr/ml after administration of a once-daily 50 mg dose of said controlled release carvedilol tablet.

75) The controlled release dosage form of claim 73 wherein said dosage form exhibits a mean Cₘₐₓ for carvedilol of about 80.01 ng/ml after administration of a once-daily 50 mg dose of said controlled release carvedilol tablet.

76) The controlled release dosage form of claim 74 wherein said dosage form exhibits a mean Cₘₐₓ for carvedilol of about 70.84 ng/ml after administration of a once-daily 50 mg dose of said controlled release carvedilol tablet.

77) The controlled release dosage form of claim 75 wherein said dosage form exhibits a median Tₘₐₓ for carvedilol of about 8.45 hr after administration of a once daily 50 mg dose of said controlled release carvedilol tablet.

78) The controlled release dosage form of claim 76 wherein said dosage form exhibits a median Tₘₐₓ for carvedilol of about 8.45 hr after administration of a once daily 50 mg dose of said controlled release carvedilol tablet.

79) The controlled release dosage form of claim 44 further comprising a coating.

80) The controlled release dosage form of claim 79 wherein said coating comprises a second drug.

81) The controlled release dosage form of claim 80 wherein said second drug is selected from the group consisting of ACE inhibitors, diuretics, digoxin and any combination thereof.

82) A method of treating a cardiovascular condition in a subject in need of such treatment comprising administering to said subject the controlled release dosage form of claim 43.

83) The method of claim 82 wherein said cardiovascular condition is selected from the group consisting of hypertension, congestive heart failure, left ventricular dysfunction following myocardial infarction and any combination thereof.

84) The method of claim 82 wherein said dosage form is administered once daily.

85) The controlled release dosage form of claim 44 further comprising a polyoxyalkylene block copolymer.

86) The controlled release dosage form of claim 1 wherein said carvedilol comprises a solid dispersion of carvedilol.

87) The controlled release dosage form of claim 44 wherein said carvedilol comprises a solid dispersion of carvedilol.
